(19) United States
(12) Patent Application Publication
(10) Pub. No.: US 2012/0245483 A1
(43) Pub. Date: Sep. 27, 2012

(54) System And Garment For Muscle Relaxation Of A Spastic Muscle

(75) Inventor: Fredrik Lundqvist, Stockholm (SE)

(73) Assignee: INERVENTIONS AB, Solna (SE)

(21) Appl. No.: 13/513,223

(22) PCT Filed: Dec. 2, 2010
(86) PCT No.: PCT/EP2010/068721

§ 371 Date: Jun. 12, 2012

Related U.S. Application Data
(60) Provisional application No. 61/283,374, filed on Dec. 3, 2009.

(30) Foreign Application Priority Data
Apr. 27, 2010 (SE) 1050420-7

(51) Int. Cl. A61N 1/36; A61H 1/00; A61B 5/0488

(52) U.S. Cl. 600/546; 601/15

(57) ABSTRACT

The present invention relates generally to muscle relaxation. Muscle relaxation is desired in many disease states, including spastic paresis and biomechanical and neuromuscular dysfunction. More specifically, the invention relates to a system that causes muscle relaxation by reducing muscular spasticity through the stimulation of joints and muscles. The system consists of a garment with electrodes, a hardware unit and software controlling the stimulation.

### TECHNICAL FIELD

The present invention relates in general to muscle relaxation, and more particular to muscle relaxation for spastic muscles in patients having injuries to the central nervous system (CNS) at least by using muscle stimulation.

### BACKGROUND

Injuries to the central nervous system (CNS) are difficult to treat and cure. Spastic paresis, which is a pathologically increased muscle tonus caused by an injury to the central nervous system (CNS) is a significant obstacle for prevention of posturing and loss of mobility.

Today, therapeutic alternatives for the reversal of CNS injury symptoms, such as spasticity, are very limited. Therapies are constructed to prevent further loss of function, rather than alleviating the symptoms. No treatment has been found to truly give back function and, in the long run, reversing the injury through muscle relaxation of spastic muscles.

In addition to the spasms themselves, musculoskeletal pain is a common related complaint. Pain originating from dysfunction in the musculoskeletal system is in most cases caused by muscle spasms due to muscular imbalance. If the pain is not treated properly, patients risk developing chronic pain syndromes, conditions that are difficult to cure.

There are several techniques available to affect muscles in the human body.

Electrical muscle stimulation (EMS), also known as neuromuscular electrical stimulation or electromyostimulation is a commonly known method for increasing muscle mass in specific areas, by providing an electric current into the muscle causing contraction, which gradually leads to increased mass in the treated muscle. Trancutaneous Electrical Nerve Stimulation (TENS) is closely related to EMS, but instead of stimulating muscles to contract, electric stimulation is used to indirectly treat pain, by distracting the brain through the stimulation of other body parts. In U.S. Pat. No. 4,580,572, a garment for electrical monitoring of sites or electrical stimulation, such as EMS is disclosed.

However, none of the currently known muscle stimulation techniques is suited to provide for targeted muscle relaxation.

Hence, a new system, and garment allowing for increased muscle relaxation would be advantageous.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a system and garment allowing for improved muscle relaxation in spastic patients.

An object of the present invention is to provide for muscle relaxation.

Furthermore, the invention relates to a system that causes muscle relaxation by reducing muscular spasticity through the stimulation of several muscles and joints simultaneously.

Furthermore, an object is to reduce muscle spasms due to spastic paresis or other dysfunction of the neuromuscular system inducing muscle spasms.

Moreover, an object is to reduce posturing and development of contractures in patients with spastic paresis.

Another object is to give spastic patients a self-sufficient rehabilitation instrument enabling increased function and movement leading to better quality of life.

Another object is to reduce pain in patients with spastic paresis and in patients with biomechanical and neuromuscular dysfunction.

Another object is to produce a powerful diagnostic tool for therapists specialized in neurology, orthopedics and manual therapy.

Another object is to be a reliable research system in neurological research regarding brain injuries.

Another object is to getting a grading or measuring scale for spasticity.

According to an aspect a system for relaxation of a spastic antagonist muscle of a human is provided. The system comprises an electronic muscle stimulation device having a first electrode and a second electrode for connection to the corresponding agonist muscle. The system further comprises a vibrator device for connection to a ligament, joint capsule, or tendon to which the agonist muscle attaches to the skeleton. Moreover, the system comprises control unit configured to simultaneously control the electronic muscle stimulation device and the vibrator device, by applying a first pulsed current signal between the first electrode and the second electrode, and a second pulsed current signal to the vibrator device.

According to another aspect a garment comprising the system is provided.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 illustrates the system according to an embodiment;

FIG. 2 illustrates the system according to another embodiment;

FIG. 3 illustrates a front view of a garment according to an embodiment;

FIG. 4 illustrates a back view of a garment according to an embodiment;

FIG. 5 illustrates a front view of the garment components according to an embodiment;

FIG. 6 illustrates a back view of the garment components according to an embodiment;

FIG. 7 illustrates a front view of EMS/EMG-electrode placement according to an embodiment;

FIG. 8 illustrates a back view of EMS/EMG-electrode placement according to an embodiment;

FIG. 9 illustrates a front view of the vibration electrode placement according to an embodiment;

FIG. 10 illustrates a back view of the vibration electrode placement according to an embodiment;

FIG. 11 illustrates a front view of all anatomic electrode placements according to an embodiment;

FIG. 12 illustrates a back view of all anatomic electrode placements according to an embodiment; and

FIG. 13 illustrates a front view of garment components, hardware placement, connection ports and cable bundle connectors according to an embodiment.

### DESCRIPTION OF EMBODIMENTS

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the description of the particular embodiments illustrated in the accompanying drawings is not intended to be limiting of the invention.

An idea is to combine EMS stimulation and joint tissue vibration stimulation for patients with spastic paresis and/or musculoskeletal pain in order to achieve improved relaxation in the muscle/muscles being spastic.

The combination of EMS stimulation and joint tissue vibration stimulation may also be utilized to decrease inflammation, decrease muscle tension and building muscles to treat imbalance, by utilizing a combination of EMS stimulation for a muscle and vibration stimulation for a joint or tendon correlated to the muscle.

The present inventor has realized that by utilizing the generally concept of antagonist muscle pairs, together with combination of EMS stimulation and joint tissue stimulation, spastic muscles may be relaxed when the stimulation is performed in a certain way. This was done by manual stimulation of patients joint tissues, combined with EMS stimulation. The present inventor has surprisingly found that the combination of simultaneous EMS stimulation and joint tissue vibration gives far better results in terms of relaxation of spastic muscles than utilizing EMS stimulation, and vibration stimulation, separately. By performing the manual stimulation according to above, a number of specific advantageous combinations were found, which is described below.

Specifically, it was discovered that stimulation may have a systemic effect, i.e. that stimulation of a single muscle and joint or tendon correlated to the muscle, or a group of adjoining muscles and joints or tendons correlated to the respective muscles may affect muscles in a different place of the body.

Antagonistic pairs are needed in the body because muscles can only exert a pulling force, and can not push themselves back into their original positions. An example of this kind of muscle pairing is the biceps brachii and triceps brachii. When the biceps are contracting, the triceps are relaxed, and stretches back to its original position. The opposite happens when the triceps contract.

Agonist is a classification used to describe a muscle that causes specific movement or possibly several movements to occur through the process of its own contraction. Each antagonist pair comprises an agonist muscle and an antagonist muscle. Hence, when the biceps brachii is contracted, it acts as the agonist muscle, and the triceps brachii will act as an antagonist muscle. On the other hand, when the triceps brachii is contracted, it acts as the agonist muscle, and the biceps brachii will act as an antagonist muscle.

The present inventor has found that mild agonist muscle stimulation leads to reciprocal inhibition of the antagonist muscle and slight contraction without shortening of the agonist muscle. The joint tissue stimulation facilitates the agonist activation and relaxes the antagonist muscle through reciprocal inhibition. The concept consists of co-stimulation of several muscles and joint tissues simultaneously to induce muscle relaxation in a group of spastic muscles. The idea behind the muscle stimuli is to easily stimulate the agonist muscle without shortening it. The nervous system senses the stimulation, whereby the antagonist muscle experiences so called reciprocal inhibition and is prolonged due to relaxation, which indirectly leads to a shortening of the stimulated agonist muscle.

Prolonged stimulation leads to a general muscle relaxation and reduced muscle spasms in the whole body. Weak muscles are made stronger which in the long run leads to a generalized reduction of muscle spasms and musculoskeletal pain; therefore reducing disability and suffering.

EMS+Vibration

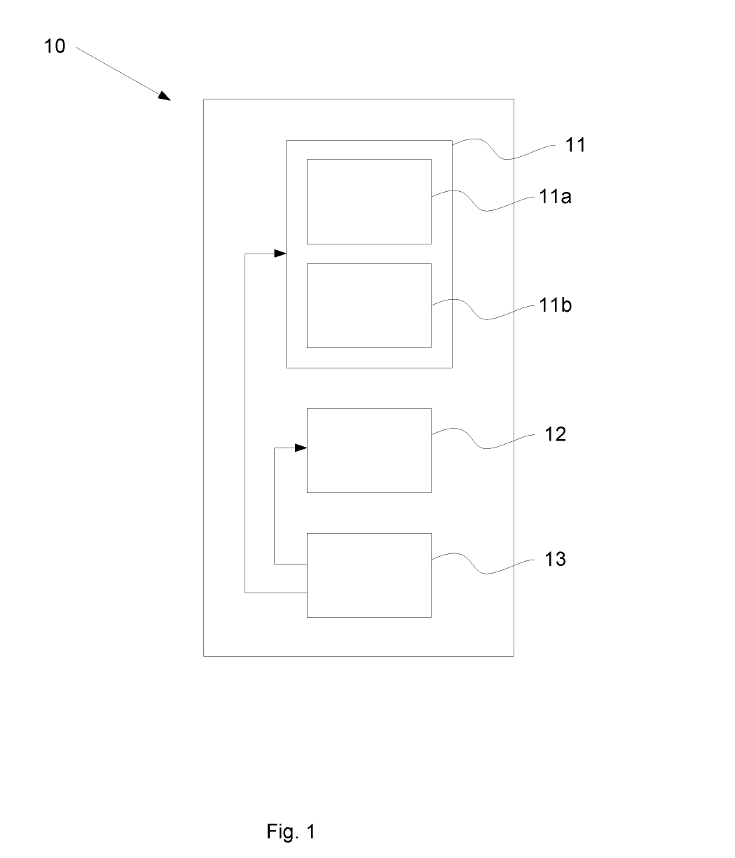

In an embodiment, according to FIG. 1, a system 10 for relaxation of a spastic antagonist muscle of a human is provided. The system comprises an electronic muscle stimulation device 11 having a first electrode 11a and a second electrode 11b for connection to the corresponding agonist muscle. The system 10 further comprises a vibrator device 12 for connection to a ligament, joint capsule, or tendon to which the agonist muscle attaches to the skeleton. Moreover, the system comprises a control unit 13 configured to control the electronic muscle stimulation device 11 and the vibrator device 12, by applying a pulsed EMS current signal between the first electrode 11a and the second electrode 11b, and a pulsed vibrator current signal to the vibrator device 12.

Usually, patients having CNS injury symptoms, has several spastic muscles, which heavily limits their mobility and life quality. According to an embodiment the system comprises an electronic muscle stimulation device or a pair of first and second electrodes for each agonist of each spastic antagonist muscle to be treated, and a vibrator device for each corresponding ligament, joint capsule, or tendon to each agonist muscle of each spastic antagonist muscle to be treated.

In an embodiment, the system comprises a first electrode and second electrode for the majority of the agonist muscles in the human body, as well as a vibrator device for each of the agonist muscles. The present inventor has realized that by utilizing a system providing the majority of the antagonist pair muscles in the human body with the EMS electrodes, as well as the corresponding joint tissue with vibrator devices, even though not all of the majority of muscles in the body are spastic, an increased relaxation for the truly spastic muscles is achieved, by stimulation of the majority of the muscles. This is believed to be the result of the nerve inhibiting signal substances being released to the synapses and the cerebrospinal fluid, circulating the brain and spinal cord. Therefore other synapses and neurons can be affected if in proximity of either the site of release or the cerebrospinal fluid. It is e.g. commonly known that decreased spasticity in the legs leads to decreased spasticity in the arms. The first electrode 11a and/or second electrode 11b may be any known EMS electrode suitable for the purpose of muscle relaxation, and allowing for reduced discomfort for the patient. Each of the first electrode 11a or second electrode 11b acts as a +/−node and is designed to electrically stimulate the muscle to which it is connected.

The size of the first 11a and/or second 11b EMS electrode is selected based on the muscle to be treated.

In an embodiment, the first and/or second electrode may be directly attached to the skin by means of an adhesive, such as conductive pads or conductive gel.

The electrodes may e.g. be silicone electrodes, combined with conductive gel. Important properties for electrodes are good skin contact/adhesiveness, good conductivity, hypoallergenic properties and durability.

Pulsed EMS Current Signal

In general, the parameters of the EMS current signal may be chosen which resemble the physiology of the body. The signals in the nervous system may be compared to current impulses (stimuli) to the synapses. When a certain amount of stimuli has occurred, signal substances are excreted.

Generally, a phasic EMS-stimulus is given with a frequency ranging between 2 and 50 Hz, with a duration between 5 to 300 microseconds.

Muscle relaxation in spastic muscles gives the possibility to induce controlled functional muscle contraction in chosen relaxed muscles. The frequency needed to induce muscle contraction is higher than the frequency used for optimal antagonist muscle relaxation (20 Hz/30 micros). Stimulation frequencies for functional muscle contraction are ranging from 25 to 50 Hz and the duration needed is between 50-300 micros.

The pulsed EMS current signal is controlled by at least the following parameters; pulse frequency, pulse duration, pulse strength.

Experiments have shown that muscles start to contract at a pulse frequency of approximately 15 Hz to approximately 35 Hz, at which frequency range the central nervous system feels the presence of the current signal. The present inventor has realized that by choosing a frequency as low as possible, but still detectable by the central nervous system, the discomfort for the patient is reduced, while the automatic relaxation of the spastic antagonist muscle is taken care of by the central nervous system. A higher frequency than approximately 35 Hz would lead to shortening of the stimulated agonist muscle and therefore activation of the stretch reflex in the antagonist muscle which is not desired, since this would lead to a reciprocal spasm of the agonist muscle.

The pulse duration of the current signal is selected such that it resembles the pulse duration of nervous signals. For example, a pulse duration of approximately 5 to 60 microseconds, such as 30 μs, has been found to be suitable. However, even shorter pulse duration could be advantageous. Too long pulse duration of the EMS current signal does not correspond to the neurophysiologic parameters of the body. Furthermore, longer pulse duration may also increase the risk of muscle shortage, which is not desired.

In an embodiment, the pulsed EMS current signal has a pulse frequency ranging between 10 and 30 Hz with a pulse duration ranging between 5 to 60 microseconds.

According to a preferred embodiment the pulsed EMS signal has a pulse frequency of 20 Hz, with pulse duration of 30 microseconds.

The EMS current signal strength is selected such that it does not exceed the amplitude at which the skin adjacent to the first 11a and/or second 11b EMS electrodes starts to vibrate. In use, the vibrating, pain free sensation may be felt by some patients. Stronger current signals may produce muscle shortening and pain in the patient, which is not desired. Preferably the current signal strength is selected to lie in the range of 50% to 75% of the signal strength required to feel the vibration in the skin adjacent the first and/or second electrode, in use. Hence, the pulsed EMS current signal strength does not provide discomfort for the patient, however a sparkling or tingling sensation may be felt in some patients.

### Vibration Device

In an embodiment, the vibration device 12 is a micro vibration motor, designed to stimulate joint proprioception in joint to which is in contact. The vibration device is small, round, cylindrical and covered by rubber to enhance friction with skin and therefore directing the vibration stimuli to joint tissues underlying the skin.

According to an embodiment the pulsed vibrator current signal has a pulse frequency ranging between 5 Hz to 400 Hz.

Vibration stimulus is given in three primary frequencies, designated to stimulate three important sensory receptors. One frequency is chosen to stimulate Pacinian corpuscles, one frequency is chosen to stimulate Merkel's disk receptors and one frequency is chosen to stimulate Meissner's corpuscles. The frequency range chosen for stimulating Merkel's disk receptors is 5-15 Hz. The frequency for Meissner's corpuscles is ranging between 20-50 Hz and for Pacinian corpules the stimulation frequency ranges between 60-400 Hz. Optimal vibration is defined as the mean of these ranges.

EMG

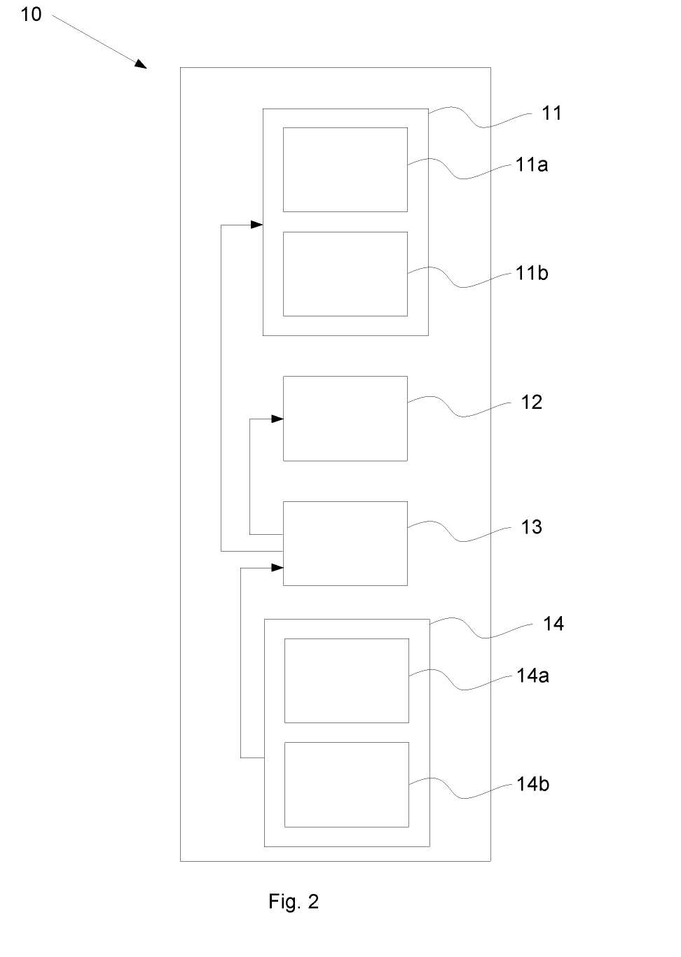

In an embodiment according to FIG. 2, the system 10 further comprises an Electromyography (EMG) device 14 for evaluating and recording the electrical activity in the spastic antagonist muscles.

Based on the electrical activity in the spastic antagonist muscle, before and/or during EMS stimulation and vibration stimulation, the parameters of the EMS current signal or vibration current signal may be adapted.

The EMG device 14 comprises a first EMG electrode 14a and a second EMG electrode 14b for each muscle for which the electrical activity is to be monitored.

The EMG electrode may be of the same type as the EMS electrodes 11a, and 11b. Hence, in an embodiment, the first 11a, and/or the second 11b electrode of the electronic stimulation device, may each act as an EMG electrode for together detecting electric signals in the muscle to which they are connected.

In use, the EMG-electrodes 14a, 14b are placed to be in contact with the spastic antagonist muscle, whereas the EMS electrodes 11a, 11b are to be placed in contact with the corresponding agonist muscle.

Calibration

Since the spastic muscle behavior in CNS injured patients differs greatly, the professional skills of a neuromuscular system specialist is required for calibrating the system before use, such that the correct agonist muscles are provided with EMS electrodes and joints corresponding thereto are provided with vibrator devices. Every chosen muscle stimulation is paired with an anatomically relevant joint stimulation in order to strengthen the desired relaxation effect. Furthermore, the parameters of the pulsed EMS current signal need to be selected, which parameters may differ between patients.

According to a non limiting theory of the inventor, the current level may need individual adjustment, since some muscles lies deeper than others. However, the frequency and pulse duration of the EMS current signal may be more or less independent of individual spasticity level, since the nervous system does not vary very much between patients.

Relaxation is initially induced by antagonist muscle stimulation by the therapist. The therapist is aided by EMG-readings showing the relaxation of spastic key muscles. The process is called spasticity calibration. The amount of stimuli needed (current×time, I×t) gives the amount of energy needed to induce reciprocal inhibition of the antagonist muscle. The easier the reciprocal inhibition is induced the lower is the severity of spasticity. Therefore the therapist can, aided by this process, establish the exact amount of stimuli needed for every patient.

During calibration, at least one muscle is chosen for spasticity calibration, e.g. based on the readings from the EMG device connected to said muscle. The muscle read by the EMG device may be the spastic antagonist muscle.

It is commonly known that spasticity in one muscle, may give rise to spasticity in another muscle. Hence, a decrease in spasticity in the legs leads to a decrease in spasticity in the arms. Hence, by reading the electrical activity in a number of muscles, during calibration, a measure of the patient's general muscle spasticity may be obtained (an indication of mass reflex activity for said patient).

According to a preferred embodiment applied to the whole body, muscles may also be chosen, such as at least three EMG-reading muscles, i.e. three muscles connected to the EMG device. For example, one pair of EMG electrodes in the arm, one pair of EMG electrodes in the leg and one pair of EMG electrodes in the spine or jaw, wherein each pair of EMG electrodes are connected to the EMG device. One EMG-electrode used for reading of non-muscular electrical surface activity may be placed in a bony area without underlying muscle to give a measurement for comparison, used for calibration.

An advantage of this embodiment is that it is possible to measure the general spasticity and/or general relaxation based on EMG readings from only a few of the muscles in the patient.

Another advantage is that the muscles being read by the EMG device may be located at some distance from the site of the intended EMS stimulated muscles, whereby the leaking of current from the EMS electrode to the EMG electrode is reduced.

In an embodiment, calibration may be performed during treatment by the system.

Control Unit

The control unit comprises a processor for running software and a memory onto which the software is stored. The control unit may be connected to a power source or a pulse generator for generating the pulsed EMS signals, and vibrator signals.

The control unit is portable, and connected with the electrodes in the garment and is able to connect to a personal computer via e.g. USB or Bluetooth. Different treatment programs and patterns can be stored in the memory. The control unit is configured to run code segments for controlling the functionality EMS device 11, vibrator device 12, and optionally the EMG device 14 of the system.

In an embodiment a computer program product is provided. The computer program product is stored on a computer-readable medium comprising the software code adapted for controlling the system when executed on a data-processing apparatus.

the code segments for controlling the system according to some embodiments.

In an embodiment the control unit is configured to send treatment information to an external device. The treatment information may, e.g. comprise information regarding the treatment progress, i.e. improvements in relaxation of the muscles. The therapist may receive this information and update the treatment strategy, change treatment plan etc.

In an embodiment, the control unit is configured to receive updated software from an external device, such as a computer. The updated software may e.g. be new treatment plans, stimulation programs received from the therapist. Hence, the system may be updated without the need for a visit at the therapist.

The external device may comprise software for performing calibration, to control EMS-programs, vibration programs and EMG-measurements enabling the spasticity calibration mode. Treatment parameters, such as EMS current signals, Vibration current signals, treatment time, muscles to be treated etc. are utilized in order to create treatment plans. The external device software may comprise a code segment for visualizing the system parameters on a display. Hence, when therapist selects a stimulation muscle and electrode and vibration stimuli placement, this is visualized on the display.

Treatment Duration

In an embodiment the treatment duration is one to two hours daily, or for a longer duration if the system is only used 2 to 4 times a week.

Garment

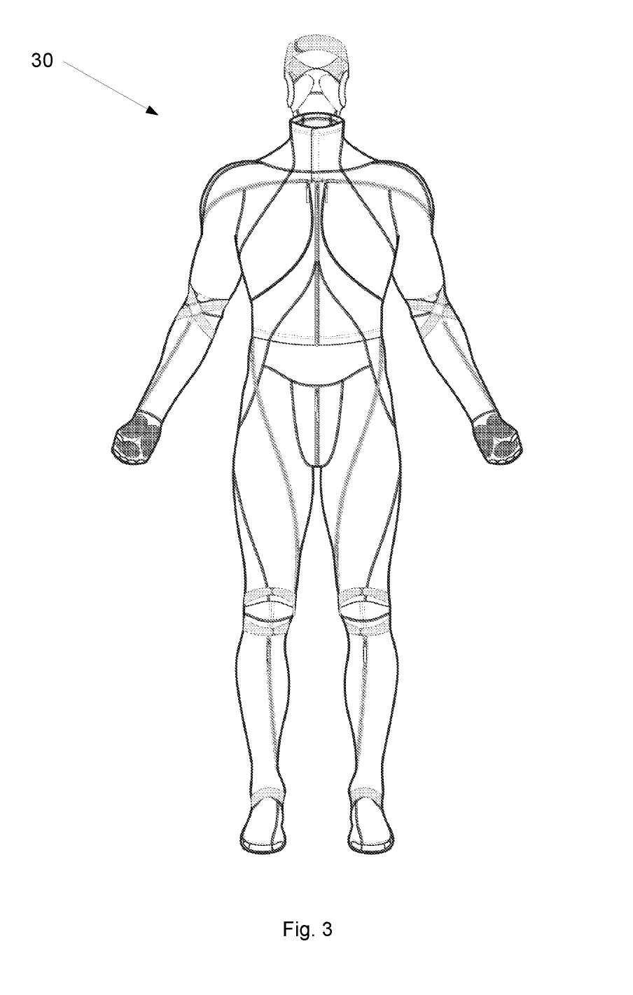

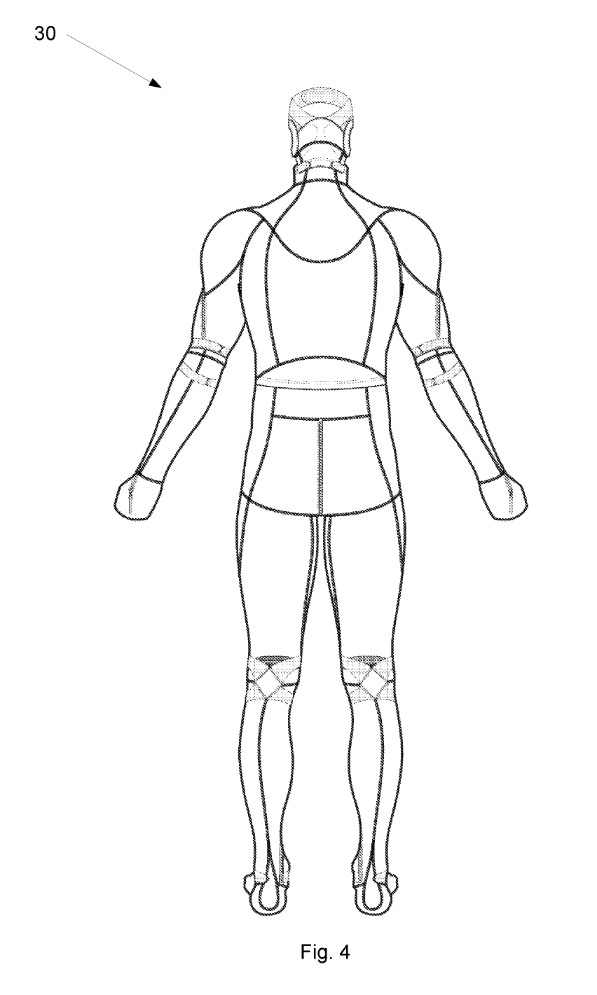

In an embodiment, according to FIGS. 3 and 4, a garment 30 comprising the system is provided. Hence, the EMS-electrodes, vibrator devices, optional EMG-electrodes and the control unit are all included in the garment 30.

The garment 30 makes it possible for a patient to receive treatment wherever he/she is without the need for an attending specialist or healthcare personnel. This greatly increases the quality of life for the patients and relatives, since no trips to the health care clinic are required, as well as the patient may retrieve treatment everywhere, as long as the garment 30 is worn.

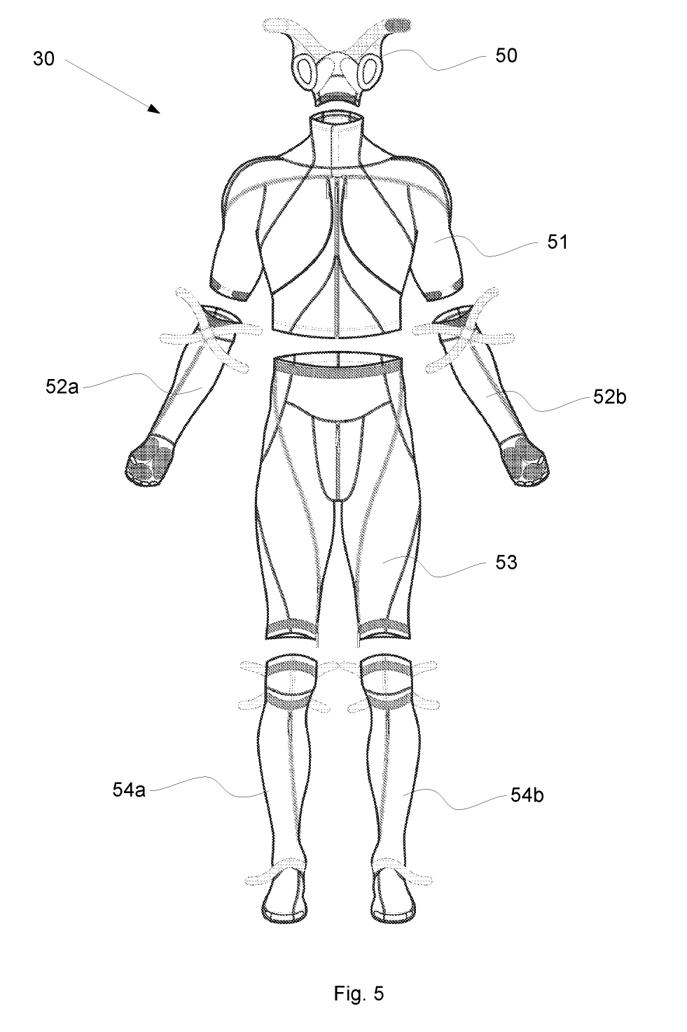

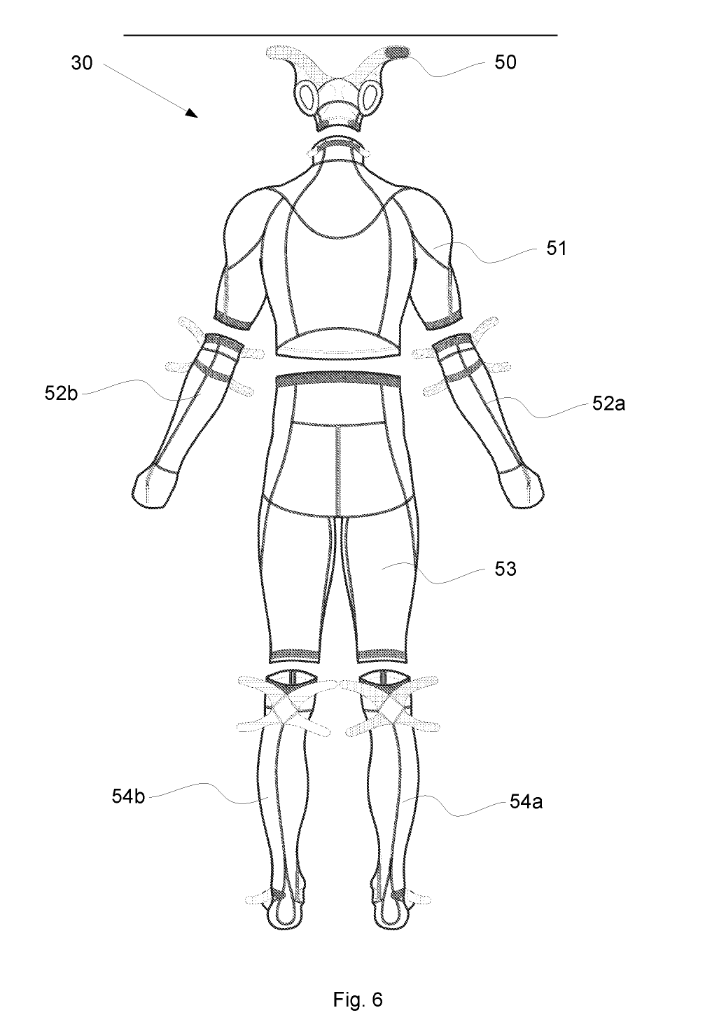

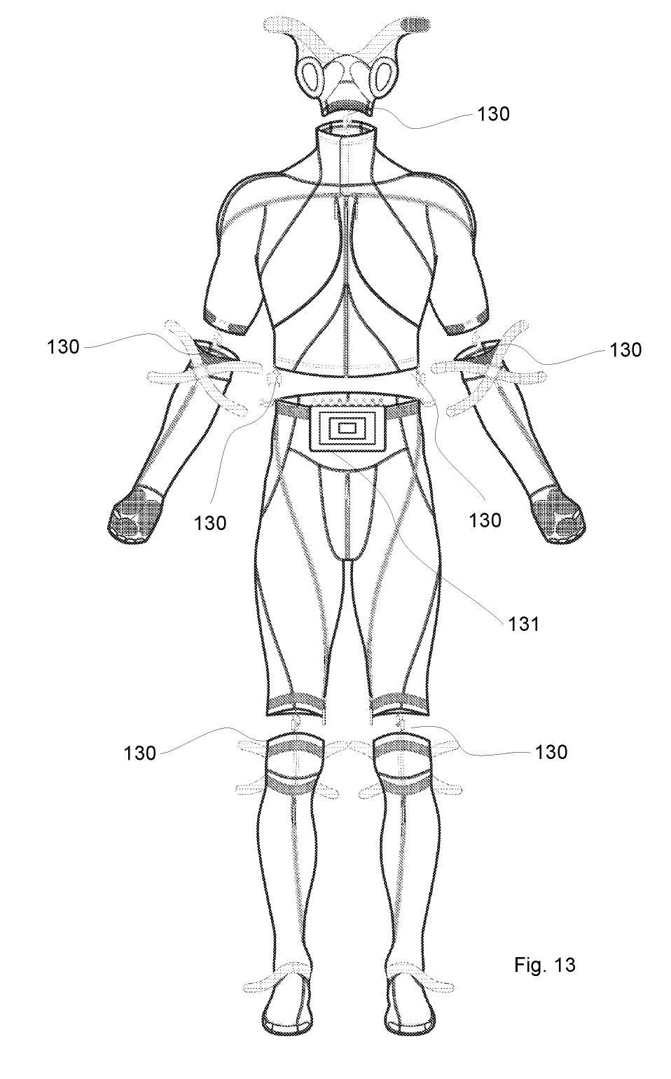

In an embodiment according to FIGS. 5 and 6, the garment 30 comprises seven interlocking parts. One part 51 for the head and neck; two parts 52a and 52b for elbow, forearm and hand; one part 53 for torso, shoulder and upper arm; one part 54 for lower back, lower abdomen, pelvis, thigh, hip and upper leg; and finally two parts 55a and 55b for knee, lower leg and foot. All parts can be connected to a hardware unit 131 separately or in different combinations, these combinations ranging from two parts to all seven parts depending on the needs of the patient. FIG. 13 illustrates the connection points 130 of the parts of the garment.

It should be appreciated that only a part of the garment, according to the previous embodiment, may be used, depending on the needs of the individual patient. Hence, for some patient there is no need for a full body garment, but one or more parts may be sufficient to allow for effective treatment.

According to an embodiment, the garment comprises of five major textile and support materials. Elastic spandex for areas covering muscles and, embedded in this spandex, muscle electrodes for skin contact; firm elastic spandex textile in joint areas to induce joint stability and specific skin contact of embedded muscle and vibration electrodes; and Velcro to interlock the garment parts and also induce joint stability and electrode skin contact. Zippers are placed in the different garment parts to enable simple dressing and use of the garment. Padding and other supportive materials are placed between the textile layers to enhance stability and electrode skin contact.

In order to provide for a perfect garment fit for each patient, each garment may be tailor made for each patient. Hence, each patient may be individually measured. Based on the calibration made by the specialist, the therapist chooses which muscles to stimulate and therefore induce muscle relaxation of corresponding spastic muscles. The tailor made garment is produced and the control unit is programmed with the necessary parameters such as to perform a vibrator and EMS stimulation in the prescribed manner.

Based on the individual measurements, a tailor made garment may be provided. Sufficient data is sent to a factory to ensure tailor made production and delivery of a functional garment. Final tailoring (e.g. minor adjustments of the garment), calibration and hardware unit programming may be performed after construction and delivery of the garment.

In another embodiment, the garment may be chosen from a big variety of garment component sizes which are combined to fit all different possible size requirements.

The design of the anatomical measurement charts enables exact anatomical positioning of electrodes stimulating specific muscles.

The type of the first and second electrodes for each muscle may be selected based on muscle type, and their location in the garment, such as to avoid discomfort for the person being treated. Essentially two types of EMS-electrodes may be used.

The first EMS-electrode type is round, soft, dry, convex, pointing to the skin from the inside of the garment and through the elasticity of the spandex pressed against the skin without discomfort. Different electrode sizes are available for different sizes of muscles to be stimulated.

The second EMS-electrode type is square, soft, dry and flat for areas prone to pressure e.g. the buttocks area (sitting) or areas with overlying firm spandex or Velcro. Different electrode sizes are available for different sizes of muscles to be stimulated.

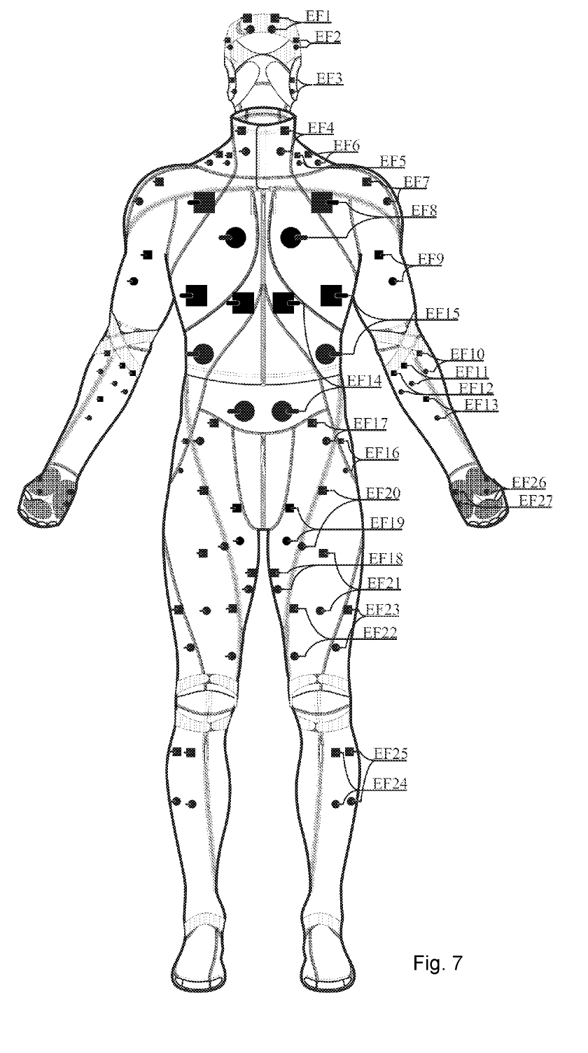

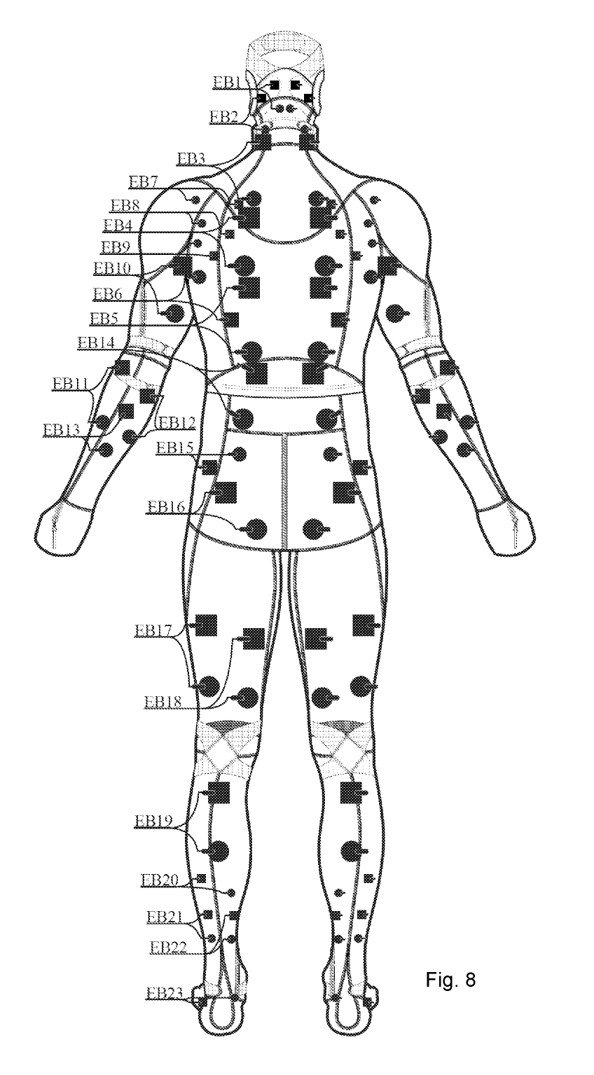

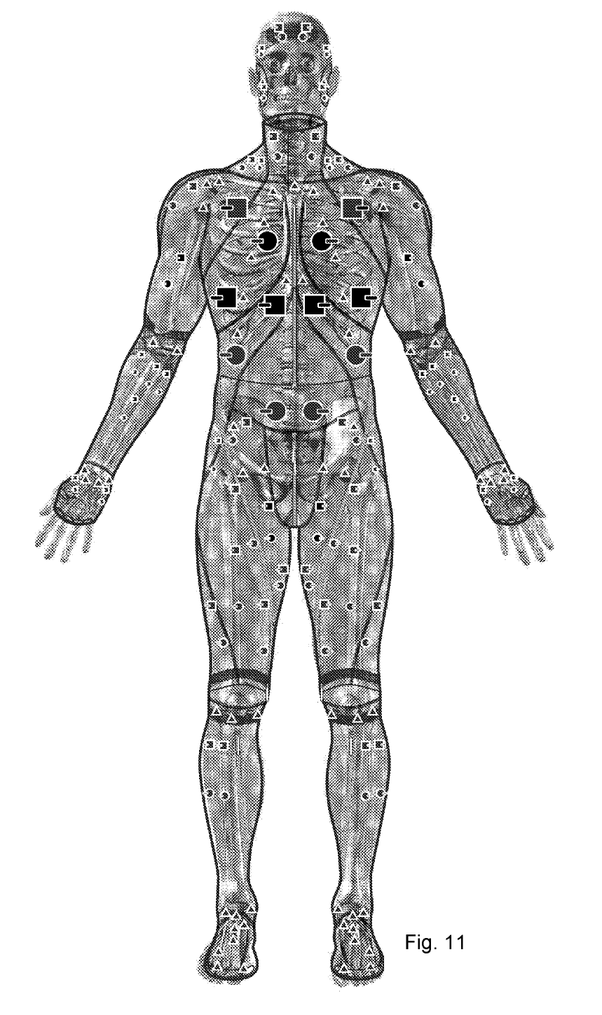

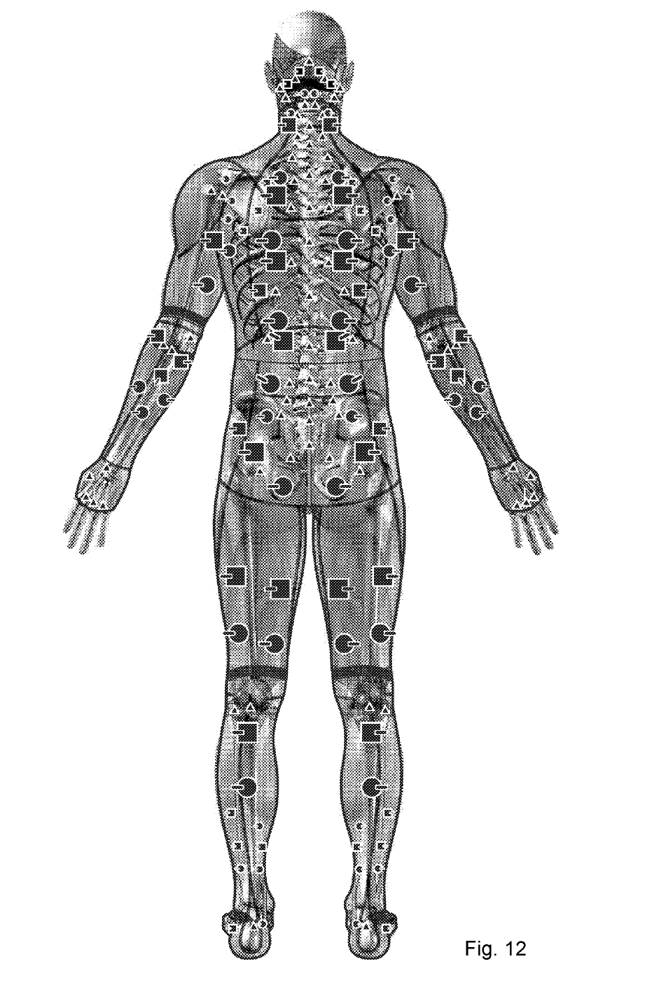

200 possible anatomical EMS-electrode placements are marked in FIG. 7, FIG. 8, FIG. 11 and FIG. 12. The EMG-electrodes are only used clinically by the therapist when calibrating stimulation patterns. EMG-analysis is possible in the same locations as for the EMS-electrodes (200 positions) in FIG. 7, FIG. 8, FIG. 11, FIG. 12.

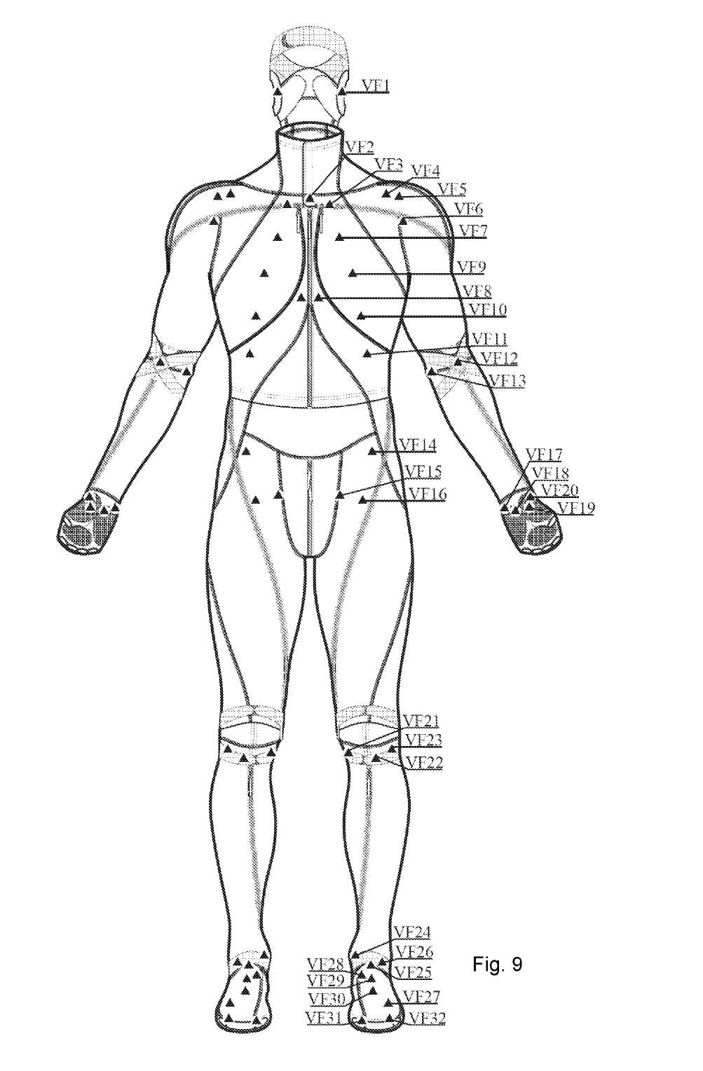

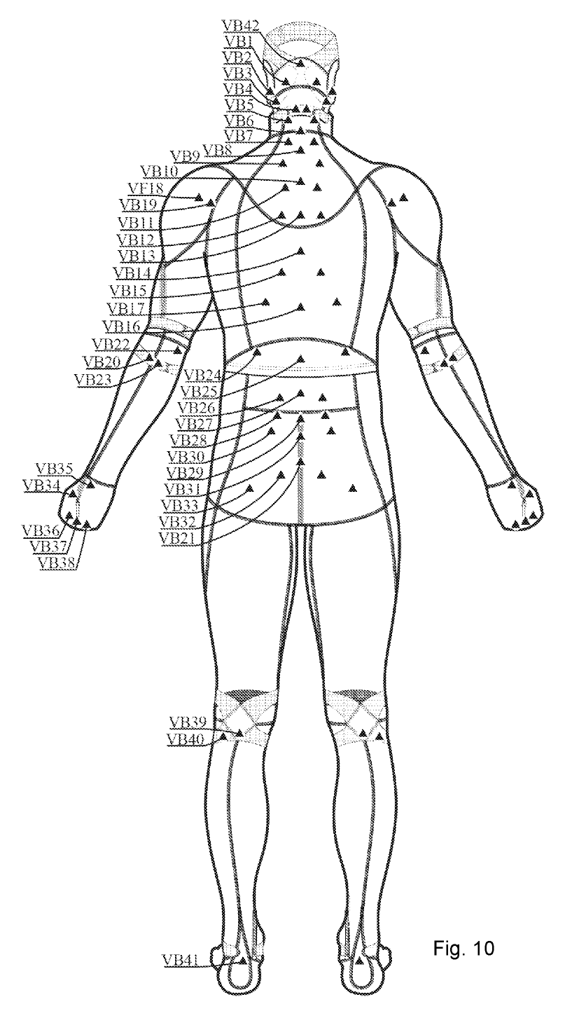

The vibration electrodes are pressed against the skin with the help of firm spandex and Velcro in the garment. The positioning of the vibration electrodes in the garment is anatomically and therefore physiologically specific. 137 possible anatomical vibration-electrode placements are marked in FIG. 9, FIG. 10, FIG. 11. and FIG. 12.

Optionally, EMG electrodes are placed in the garment to monitor the muscle relaxation in the spastic muscles.

The therapist is able to send a treatment program to the control unit via Internet if needed. The control unit is very easy to use; the patient chooses a program and initiates suitable stimulation. The control unit may be connected to the different garment components through a maximum of nine cable bundle connection ports 130, see FIG. 13. Five connections are positioned on the top of the hardware unit 131 supplying head, torso and arm components with stimuli. FIG. 13 Four connection ports may be placed on the bottom of the hardware unit 131 supplying the pelvic component and the two leg components in the garment, see FIG. 13.

The hardware unit 131 may be placed in a pouch on the umbilical area. This hardware unit placement enables connection ports to be in a suitable position for port connection of cable bundles originating in different body parts, see FIG. 13.

Pairing Charts

The muscles to be stimulated for each patient are chosen by the therapist during calibration of the system or garment. The stimulation muscles chosen by the therapist are the muscles “losing” against their stronger spastic antagonists.

Each agonist muscle, to which EMS stimulation is to be delivered, is paired with anatomically relevant joints, which will receive vibration stimulation by means of the vibrator device.

The muscles suitable for stimulation may be identified by a therapist. In an embodiment, the therapist identifies the muscles which are inferior to a stronger/shortened antagonist, i.e. the muscles which are counteracting the erroneous position. For example, in a straightened spastic leg (knee), the back of the thigh, which may act to bend the leg by the knee, may be identified as a target for stimulation to bring the leg out of its spastic position. The identification of muscles for stimulation may result in pairing charts, for example according to below.

FIGS. 7 to 10 each illustrate different possible electrode placements.

In FIGS. 7 to 10 “M” stands for “muscle electrode pair for EMS/EMG”, “F” stands for front, “B” stands for back.

FIG. 7 illustrates locations for the first and second EMS electrode for different muscles on the front side of the body, wherein the reference numbers correspond to the muscle according to the table 1 below.

TABLE 1

| Reference | |
|---|---|
| number | Muscle |
| EF1 | Occipitofrontalis muscle |
| EF2 | Temporalis muscle |
| EF3 | Masseter muscle |
| EF4 | Sternocleidomastoideus muscle |
| EF5 | Scalenius muscles |
| EF6 | Superior Trapezius muscle |
| EF7 | Pectoralis major muscle |
| EF8 | Deltoideus muscle |
| EF9 | Biceps brachi muscle |
| EF10 | Brachioradialis muscle |
| EF11 | Flexor carpi radialis muscle |
| EF12 | Flexor carpi ulnaris muscle |
| EF13 | Flexor digitorum superficialis and profundus muscles |
| EF14 | Rectus abdominis muscle |
| EF15 | Obliqus externus muscle |
| EF16 | Tensoror fascia latae muscle |
| EF17 | Iliacus muscle |
| EF18 | Adductor longus muscle |
| EF19 | Adductor magnus muscle |
| EF20 | Sartorius muscle |
| EF21 | Rectus femoris muscle |
| EF22 | Quadriceps medial vastus muscle |
| EF23 | Quadriceps lateral vastus muscle |
| EF24 | Tibialis anterior muscle |
| EF25 | Fibularis longus muscle |
| EF26 | Thenaris muscle |
| EF27 | Hypothenaris muscle |

As may be observed from Table 1, 27 front muscles, on each bilateral side of the body, has been identified for optional placement of the EMS electrodes.

FIG. 8 illustrates locations for the first and second EMS electrode for different muscles on the back side of the body, wherein the reference numbers correspond to the muscle according to the Table 2 below.

TABLE 2

| Reference | |
|---|---|
| number | Muscle |
| EB1 | Suboccipital muscles |
| EB2 | Splenius capitis and cervicis muscle, superior cervical |
| | erector spine muscles |
| EB3 | Medial and inferior trapezius muscle, inferior cervical erector |
| | spine muscles, superior thoracal erector spine muscles |
| EB4 | Midthoracal erector spine muscles |
| EB5 | Thoracolumbal erector spine muscles |
| EB6 | Latissimus dorsi muscle |
| EB7 | Infraspinatus muscle |
| EB8 | Teres minor muscle |
| EB9 | Teres major muscle |
| EB10 | Triceps brachii muscle |
| EB11 | Extensor carpi radialis and supinator muscle |
| EB12 | Extensor carpi ulnaris muscle |
| EB13 | Extensor communis digitorum and (pollicis, digiti minimi) |
| | muscle |
| EB14 | Quadratus lumborum and lumbal erector spine muscles |
| EB15 | Gluteus medius muscle |
| EB16 | Gluteus maximus muscle |
| EB17 | Biceps femoris muscle |
| EB18 | Semimembranosus and semitendinosus muscle |
| EB19 | Gastrocnemius muscle |
| EB20 | Soleus muscle |
| EB21 | Flexor digitorum longus muscle |
| EB22 | Flexor hallucis longus muscle |
| EB23 | Plantar pedis muscles |

As may be observed from Table 2, 23 back muscles, on each bilateral side of the body has been identified for placement of the EMS electrodes. Hence, in total 50 muscle groups, i.e. 23 back muscles and 27 front muscles, has been identified for optional placement of the EMS electrodes/EMG electrodes. This means in total 100 possible muscles to be stimulated in the whole body.

Placement of 2 electrodes for every muscle gives a maximum of 200 possible EMS/EMG-electrode placements. Each EMS electrode placement is also a possible placement site for EMG-measurement.

FIG. 9 illustrates locations for the vibrator device on the front side of the body, wherein the reference numbers correspond to the position, ligament, joint capsule, or tendon according to the table 3 below.

TABLE 3

| Reference | |
|---|---|
| number | Position, ligament, joint capsule, or tendon |
| VF1 | Under the distal zygomatical arch anterior of the mandibular |
| | condylary process, at joint capsule of the temporomandibular |
| | joint and proximal to the temporomandibular ligament |
| VF2 | Interclavicular ligament and anterior sternoclavicular ligament |
| VF3 | Costoclavicular ligament and posterior sternoclavicular |
| | ligament |
| VF4 | Anterior acromioclavicular ligament and coracoacromial |
| | ligament |
| VF5 | Coracoclavicular ligament |
| VF6 | Anterior glenohumeral ligament |
| VF7 | Intercostal region of 3rd and 4th rib and costochondral joints |
| | of ribs 3 and 4 |
| VF8 | Costoxiphoidal ligament |
| VF9 | Intercostal region of 5th and 6th rib, and costochondral joints |
| | 5 and 6 |
| VF10 | Intercostal region of 7th and 8th rib and costochondral joints |
| | 9 and 10 |
| VF11 | Intercostal region of 9th and 10^{th} rib and costochondral joints |
| | 9 and 10 |
| VF12 | Anterior articular joint capsule of humeroulnar joint |
| VF13 | Collateral ulnar ligament in the humeroulnar joint |
| VF14 | Anterior superior iliac spine |
| VF15 | Inguinal ligament |
| VF16 | Pubofemoral ligament |
| VF17 | Ulnocarpeal ligament |
| VF18 | Radiocarpeal ligament |
| VF19 | Radiocarpeum ligament |
| VF20 | Collateral radial ligament in the radiocarpeal joint |
| VF21 | Collateral tibiofemoral ligament |
| VF22 | Anterior cruziate ligament and patellar tendon |
| VF23 | Collateral fibulofemoral ligament |
| VF24 | Deltoideum ligament |
| VF25 | Dorsal cuboideo- and cuneonavicular ligament |
| VF26 | Calcaneofibular ligament and lateral talocalcaneal ligament |
| VF27 | Peroneus brevis tendon at insertion of proximal metatarsal |
| | bone 5 |
| VF28 | Tibialis anterior tendon, plantar insertion at the navicular bone |
| VF29 | Peroneus longus tendon, plantar insertion at the cuboid bone |
| VF30 | Plantare longum ligament |
| VF31 | Transversal metatarsal ligament between toes I and II, |
| | dorsal joint capsule of metatarsophalangeal joints |
| | of big toe and second toe |
| VF32 | Transversal metatarsal ligament between toe 4 and 5 |
| VF33 | Sphenozygomatical suture and ligaments |

FIG. 10 illustrates locations for the vibrator device on the back side of the body, wherein the reference numbers correspond to the position, ligament, joint capsule, or tendon according to the table 4 below.

TABLE 4

| Reference | |
|---|---|
| number | Position, ligament, joint capsule, or tendon |
| VB1 | Atlantooccipital membrane |
| VB2 | Mastoid process |
| VB3 | Posterior to the mandibular condylary process, in proximity to |
| | the transversal process of atlas and the stylomandibular |
| | ligament |
| VB4 | Lateral spinous process of axis (C2) |
| VB5 | Transversal process of C3 |
| VB6 | Spinous process of C5 |
| VB7 | Transversal process of C5 |
| VB8 | Spinous process of prominens (C7) |
| VB9 | Transversal process Th 2 |
| VB10 | Spinous process of Th 3 |
| VB11 | Transversal process of Th 4 |
| VB12 | Transversal process of Th 6 |
| VB13 | Spinous process of Th 5 |
| VB14 | Spinous process of Th 7 |
| VB15 | Transversal process of Th 9 |
| VB16 | Spinous process of Th 10 |
| VB17 | Angule of 10^{th} rib |
| VB18 | Posterior glenohumeral ligament |
| VB19 | Posterior joint capsule in humeroulnar joint |
| VB20 | Collateral radial ligament in humeroradial/radioulnar joint |
| VB21 | Spinous process of S3 |
| VB22 | Posterior joint capsule of humeroulnar joint |
| VB23 | Posterior anulare radi ligament |
| VB24 | End of 12th rib |
| VB25 | Spinous process of L1 |
| VB26 | Transversal process of L4 and superior iliolumbal ligament |
| VB27 | Spinous process of L3 |
| VB28 | Transversal process of L5 and inferior iliolumbal ligament |
| VB29 | Spinous process of L5 |
| VB30 | Posterior superior iliac spine |
| VB31 | Spinous process of S1 |
| VB32 | Sacral insertion of sacrospinal ligament and sacrotuberal |
| | ligament |
| VB33 | Ischiofemoral ligament |
| VB34 | Posterior metacarpophalangeal joint of thum |
| VB35 | Intercarpeum arcuatum ligament and dorsal radiocarpeum |
| | ligament |
| VB36 | Distal intermetacarpal region of metacarpal bones 2 and 3 |
| VB37 | Distal intermetacarpal region of metacarpal bones 3 and 4 |
| VB38 | Distal intermetacarpal region of metacarpal bones 4 and 5 |
| VB39 | Posterior cruziate ligament and posterior joint capsule of |
| | knee joint |
| VB40 | Posterior tibiofibular ligament |
| VB41 | Achilles tendon at insertion of calcaneal bone |
| VB42 | Occipital protuberantia |

As may be observed from Table 3 and 4, in total 75 vibration sites, whereof 62 are bilateral, and 13 are located along the midline. This gives in total 137 (62*2+13) possible vibration sites of the human body, for optional placement of the vibrator devices.

According to an embodiment, a first pairing chart, illustrated in table 5 below, pairing each agonist muscle with a number of relevant joints, ligaments or tendons, is provided. The pairing chart pairs may be used as a valuable tool for the specialist calibrating the system and/or garment.

TABLE 5

| Agonist | |
|---|---|
| Muscle | Position, ligament, joint capsule, or tendon |
| EF1 | VF33 |
| EF2 | VF1, VB3 |
| EF3 | VF1, VB3 |
| EF4 | VB3, VB42 |
| EF5 | VB5, VB7 |
| EF6 | VB4, VB5, VB7, VB8 |
| EF7 | VF2, VF3, VF4, VF5, VF6, VF7 |
| EF8 | VB18 |
| EF9 | VB23, VF12 |
| EF10 | VF12 |
| EF11 | VF19 |
| EF12 | VF13, VF18, VF20 |
| EF13 | VF17, VF18 |
| EF14 | VF8, VF9, VF10, VF11 |
| EF15 | VF8, VF9, VF10, VF11 |
| EF16 | VF21 |
| EF17 | VF15, VF14 |
| EF18 | VF16, VF23 |
| EF19 | VF16, VF23 |
| EF20 | VF15, VF21 |
| EF21 | VF14, VF15 |
| EF22 | VF22 |
| EF23 | VF22 |
| EF24 | VF24, VF25, VF27, VF28, VF32 |
| EF25 | VF32, VF26 |
| EF26 | VF17, VF18, VF19 |
| EF27 | VF17, VF18, VF19 |
| EB1 | VF1, VB1, VB2, VB42 |
| EB2 | VB4, VB5, VF1 |
| EB3 | VB6, VB5, VB7, VB8, VB9, VB10, VB11 |
| EB4 | VB12, VB13, VB14, VB15 |
| EB5 | VB16, VB17, VB24, VB25 |
| EB6 | VB18, VB16, VB17, VB24, VB25 |
| EB7 | VB18 |
| EB8 | VB18 |
| EB9 | VF6 |
| EB10 | VB19, VB22 |
| EB11 | VB20, VB23, VB34, VB35, VB36, VB37, VB38 |
| EB12 | VB20, VB34, VB35, VB36, VB37, VB38, VF20 |
| EB13 | VB34, VB36, VB37, VB38 |
| EB14 | VB26, VB27, VB28, VB29, VB30, VB31, VB32, VB33 |
| EB15 | VB28, VB29, VB30, VB31, VB32, VB33 |
| EB16 | VB30, VB32, VB33 |
| EB17 | VB39, VB40 |
| EB18 | VB39 |
| EB19 | VB41, VF29, VF31 |
| EB20 | VB41, VF29, VF30, VF31 |
| EB21 | VF31 |
| EB22 | VF30 |
| EB23 | VF30 |

In another embodiment, a second pairing chart, illustrated in table 6 below, is provided, pairing a number of agonist muscles with a number of relevant joints, ligaments or tendons, i.e. several muscle and vibration stimulations simultaneously to induce general change in posture in extremities or in the rest of the body, including the spine and head.

TABLE 6
Intermuscular pairing for coupled joint motion stimulation

| | | |
|---|---|---|
| | | Position, ligament, |
| Anatomical part of the | | joint capsule, or |
| body (functionality) | Agonist muscle | tendon |

Head/Neck

| | | |
|---|---|---|
| Flexion | EF4 | VB3, VB42, bilateral |
| Extension | EB1 | VF1, VB1, VB2, VB42, |
| | | bilateral |
| | EB2 | VB4, VB5, VF1, |
| | | bilateral |
| Rotation | EF4 | VB3, VB42, |
| | | contralateral (the muscle |
| | | of stimuli is on the |
| | | oposiote side in relation |
| | | to the movement. E.g. |
| | | stimulus of left |
| | | sternocleido gives right |
| | | rotation of head) |
| | EB1 | VF1, VB1, VB2, VB42, |
| | | ipsilateral (stimulus on |
| | | the same side as the |
| | | rotation direction) |
| | EB2 | VB4, VB5, VF1, |
| | | ipsilateral |
| Lateral flexion | EF4 | VB3, VB42, ipsilateral |
| | EF5 | VB5, VB7, ipsilateral |

Shoulder

| | | |
|---|---|---|
| Flexion | EF7 | VF2, VF3, VF4, VF5, |
| | | VF6, VF7 |
| | EF8 | VB18 |
| | EF9 | VB23, VF12 |
| Extension | EB6 | VB18, VB16, VB17, |
| | | VB24, VB25 |
| | EB9 | VF6 |
| | EB10 | VB19, VB22 |
| External rotation | EB7 | VB18 |
| | EB8 | VB18 |
| Internal rotation | EB9 | VF6 |
| | EF7 | VF2, VF3, VF4, VF5, |
| | | VF6, VF7 |
| Abduction | EF8 | VB18 |
| Adduction | EF7 | VF2, VF3, VF4, VF5, |
| | | VF6, VF7 |
| | EB6 | VB18, VB16, VB17, |
| | | VB24, VB25 |
| | EB9 | VF6 |

Elbow

| | | |
|---|---|---|
| Flexion | EF9 | VB23, VF12 |
| | EF10 | VF12 |
| Extension | EB10 | VB19, VB22 |
| Supination | EF9 | VB23, VF12 |
| | EB11 | VB20, VB23, VB34, |
| | | VB35, VB36, VB37, |
| | | VB38 |
| Pronation | EF11 | VF19 |

Wrist

| | | |
|---|---|---|
| Dorsal flexion | EB11 | VB20, VB23, VB34, |
| | | VB35, VB36, VB37, |
| | | VB38 |
| | EB12 | VB20, VB34, VB35, |
| | | VB36, VB37, VB38, |
| | | VF20 |
| | EB13 | VB34, VB36, VB37, |
| | | VB38 |
| Palmar flexion | EF11 | VF19 |
| | EF12 | VF13, VF18, VF20 |
| | EF13 | VF17, VF18 |
| Supination | EB11 | VB20, VB23, VB34, |
| | | VB35, VB36, VB37, |
| | | VB38 |
| | EF12 | VF13, VF18, VF20 |
| Pronation | EF11 | VF19 |
| | EB12 | VB20, VB34, VB35, |
| | | VB36, VB37, VB38, |
| | | VF20 |
| Radial deviation | EB11 | VB20, VB23, VB34, |
| | | VB35, VB36, VB37, |
| | | VB38 |
| | EF11 | VF19 |
| Ulnar deviation | EB12 | VB20, VB34, VB35, |
| | | VB36, VB37, VB38, |
| | | VF20 |
| Ulnar deviation | EF12 | VF13, VF18, VF20 |

Fingers

| | | |
|---|---|---|
| Flexion | EF13 | VF17, VF18 |
| Extension | EB13 | VB34, VB36, VB37, |
| | | VB38 |

Thoracic spine

| | | |
|---|---|---|
| Flexion | EF14 | VF8, VF9, VF10, VF11, |
| | | bilateral |
| Extension | EB3 | VB6, VB5, VB7, VB8, |
| | | VB9, VB10, VB11, |
| | | bilateral |
| | EB4 | VB12, VB13, VB14, |
| | | VB15, bilateral |
| | EB5 | VB16, VB17, VB24, |
| | | VB25, bilateral |
| Rotation | EF15 | VF8, VF9, VF10, VF11 |
| | EB3 | VB6, VB5, VB7, VB8, |
| | | VB9, VB10, VB11, |
| | | ipsilateral |
| | EB4 | VB12, VB13, VB14, |
| | | VB15, ipsilateral |
| | EB5 | VB16, VB17, VB24, |
| | | VB25, ipsilateral |
| Lateral flexion | EB3 | VB6, VB5, VB7, VB8, |
| | | VB9, VB10, VB11, |
| | | ipsilateral |
| | EB4 | VB12, VB13, VB14, |
| | | VB15, ipsilateral |
| | EB5 | VB16, VB17, VB24, |
| | | VB25, ipsilateral |
| | EB14 | VB26, VB27, VB28, |
| | | VB29, VB30, VB31, |
| | | VB32, VB33, ipsilateral |

Lumbar spine

| | | |
|---|---|---|
| Flexion | EF14 | VF8, VF9, VF10, VF11, |
| | | bilateral |
| Extension | EB14 | VB26, VB27, VB28, |
| | | VB29, VB30, VB31, |
| | | VB32, VB33 |
| Rotation | EB5 | VB16, VB17, VB24, |
| | | VB25, ipsilateral |
| | EB14 | VB26, VB27, VB28, |
| | | VB29, VB30, VB31, |
| | | VB32, VB33 |
| | | ipsilateral |
| Lateral flexion | EF15 | VF8, VF9, VF10, VF11 |
| | | ipsilateral |
| | EB14 | VB26, VB27, VB28, |
| | | VB29, VB30, VB31, |
| | | VB32, VB33 |
| | | ipsilateral |
| | EF14 | VF8, VF9, VF10, VF11, |
| | | ipsilateral |

Pelvis

| | | |
|---|---|---|
| Flexion | EF16 | VF21 |
| | EF17 | VF15, VF14 |
| | EF20 | VF15, VF21 |
| | EF21 | VF14, VF15 |
| | EB14 | VB26, VB27, VB28, |
| | | VB29, VB30, VB31, |
| | | VB32, VB33, ipsilateral |
| Extension | EB16 | VB30, VB32, VB33 |
| | EB17 | VB39, VB40 |
| | EB18 | VB39 |
| | EB14 | VB26, VB27, VB28, |
| | | VB29, VB30, VB31, |
| | | VB32, VB33 |
| | | Contralateral |

Hip

| | | |
|---|---|---|
| Flexion | EF17 | VF15, VF14 |
| | EF20 | VF15, VF21 |
| Flexion | EF21 | VF14, VF15 |
| Extension | EB16 | VB30, VB32, VB33 |
| | EB17 | VB39, VB40 |
| | EB18 | VB39 |
| External rotation | EB15 | VB28, VB29, VB30, |
| | | VB31, VB32, VB33 |
| | EF18 | VF16, VF23 |
| | EF19 | VF16, VF23 |
| | EF20 | VF15, VF21 |
| Internal rotation | EF16 | VF21 |
| | EF19 | VF16, VF23 |
| Abduction | EF16 | VF21 |
| | EB15 | VB28, VB29, VB30, |
| | | VB31, VB32, VB33 |
| Adduction | EF18 | VF16, VF23 |
| | EF19 | VF16, VF23 |

Knee

| | | |
|---|---|---|
| Flexion | EB17 | VB39, VB40 |
| | EB18 | VB39 |
| | EB19 | VB41, VF29, VF31 |
| Extension | EF22 | VF22 |
| | EF23 | VF22 |
| External rotation | EB17 | VB39, VB40 |
| Internal rotation | EB18 | VB39 |

Ankle/Foot

| | | |
|---|---|---|
| Dorsal flexion | EF24 | VF24, VF25, VF27, |
| | | VF28, VF32 |
| Plantar flexion | EB19 | VB41, VF29, VF31 |
| | EB20 | VB41, VF29, VF30, |
| | | VF31 |
| | EB21 | VF31 |
| | EB22 | VF30 |
| Supination | EF24 | VF24, VF25, VF27, |
| | | VF28, VF32 |
| Pronation | EF25 | VF32, VF26 |
| Flexion | EB21 | VF31 |
| Flexion | EB23 | VF30 |
| Big toe flexion | EB22 | VF30 |

The first and second pairing chart is each the true keys to successful, general relaxation of major body areas with several joint and motion units involved.

Since numerous modifications and changes will readily occur to those skilled in the art, it is not desired to limit the invention to the exact construction and operation shown and described, and accordingly, all suitable modifications and equivalents may be resorted to, falling within the scope of the invention.

### Example 1

This example demonstrates the use of the present invention in the treatment of severe spasticity. The patient was a 30-year-old male suffering from severe spasticity due to mitochondrial nerve disease, progressive since the age of 15. The patient was spastic in all limbs and the right side of the body was more spastic than the left side. Some voluntary movement in left arm and neck could be observed. The right arm was not extendable in full range of motion due to the development of contractures. A typical spastic pattern for upper motor lesions was observed. The torso and head were flexed and laterally flexed to the left. The neurogenic scoliosis of the spine was c-shaped right convex. Spasticity in the spinal musculature was primarily on the left side. The shoulders were internally rotated and adducted, the elbows were flexed and the hands were flexed and the fingers formed a first. The legs were adducted in the hip, extended in the hip and knee and the foot was plantarily flexed.

The following muscles were chosen for stimulation to induce muscle relaxation in spastic muscles in this patient:
EB1. suboccipital muscles
EB2. splenius capitis and cervicis muscle, superior cervical erector spine muscles
EB3. medial and inferior trapezius muscle, inferior cervical erector spine muscles, superior thoracal erector spine muscles

EB4. midthoracal erector spine muscles
EB5. thoracolumbal erector spine muscles
EB8. teres minor muscle
EB10. triceps brachii muscle
EB11. extensor carpi radialis and supinator muscle
EB12. extensor carpi ulnaris muscle
EB14. quadratus lumborum and lumbal erector spine muscles
EB15. gluteus medius muscle
EB17. biceps femoris muscle
EB18. semimembranosus and semitendinosus muscle
EF4. sternocleidomastoideus muscle
EF5. m. scalenius
EF8. deltoideus muscle
EF21. rectus femoris muscle
EF24. tibialis anterior muscle

Spinal muscles were stimulated bilaterally but with more strength on the right side. In the limbs the stimuli were also bilateral, but more similar in terms of strength.

Muscle electrodes were paired with vibration electrodes according to pairing chart 1:
EB1.VF1.VB1.VB2.VB42.
EB2.VB4.VB5.VF1.
EB3.VB6.VB5.VB7.VB8.VB9.VB10.VB11.
EB4.VB12.VB13.VB14.VB15.
EB5.VB16.VB17.VB24.VB25.
EB8. VB18.
EB10.VB19.VB22.
EB11.VB20.VB23.VB34.VB35.VB36.VB37.VB38.
EB12.VB20.VB34.VB35.VB36.VB37.VB38.VF20.
EB14.VB26.VB27.VB28.VB29.VB30.VB31.VB32.VB33
EB15.VB28.VB29.VB30.VB31.VB32.VB33.
EB17.VB39.VB40
EB18.VB39.
EF4.VB3.VB42.
EF5.VB5.VB7.
EF8.VB18.
EF21.VF14.VF15.
EF24.VF24.VF25.VF27.VF28.VF32

The therapist chose three of the stimulation muscles, and their antagonist muscles, to perform spasticity calibration. The chosen muscles formed agonist/antagonist muscle pairs. One muscle pair in both legs, one muscle pair in both arms and one muscle pair in the spine. Measuring in the spine was performed at the concave side of the scoliosis, which in this case was the left side.

Muscles Chosen for Spasticity Calibration:

EMS-Muscle Stimulation 20 Hz and 30 Mikros:
EB17. biceps femoris muscle bilateral
EB10. triceps brachii muscle bilateral
EB5. Thoracolumbal erector spine muscles right side

EMG-Muscles (Reading):
EF22. Quadriceps medial vastus muscle bilateral
EF9. Biceps brachi muscle bilateral
EB5. Thoracolumbal erector spine muscles left side

One EMG-electrode was placed over the bony part of the distal radius. This measurement electrode gave the therapist a reference measurement of non-muscular electric activity in the body.

Stimulation current was chosen as follows:

The therapist slowly increased current in one simulation muscle until vibration could be detected by palpating the muscle. Current was then decreased slowly until vibrations no longer could be detected. The aim was to chose a pain free stimulation force.

The same procedure was carefully repeated in all EMS-muscles.

EMG-readings were performed in short intervals and decreased electrical activity in spastic EMG-muscles was noted after a few minutes.

After calibration and measurement of the patient, the garment was constructed (including the placement of the electrodes) according to the specifications in the detailed description.

At a final calibration the garment was programmed. Stimuli were given for a duration of thirty minutes. After a few minutes the first signs of muscle relaxation and spasm reduction were noted by EMG-reading and physical examination of joint mobility. Increased function in voluntary movement of the left arm and the neck was observed. The garment was programmed to reproduce the stimuli patterns and stimuli forces chosen by the therapist. The recommendation was to use the garment three times a week for duration of one to two hours per session.

After a few months of use of the garment, a general decrease in spasticity was noted and further gains in motorfunction were made. Increase of voluntary movement was noted in spine, shoulders, arms and legs.

### Example 2

This example demonstrates the use of the present invention in the treatment of severe spasticity after a cerebrovascular incident. The patient was a 58-year-old male suffering from severe spasticity after a stroke in left medial cerebral artery. The patient had long history of heart disease, and was spastic in the limbs in the right side of the body, showing classic signs of right-sided hemiplegia. No voluntary movement in the right arm and right leg were noted. A typical unilateral spastic pattern for upper motor lesions directly after a unilateral cerebrovascular incident was observed. Spasticity in the spinal musculature was primarily found on the right side. The right shoulder was internally rotated and adducted. The right elbow and right the hand were flexed and the fingers were straight or slightly flexed. The right legs were adducted in the hip, extended in the hip and knee, and finally, the foot was plantarily flexed. The patient was able to walk with a severe limb and aided by cane.

The following muscles were chosen for stimulation to induce muscle relaxation in spastic muscles in this patient:
EB3. medial and inferior trapezius muscle, inferior cervical erector spine muscles, superior thoracal erector spine muscles, right side only
EB4. midthoracal erector spine muscles, bilateral, left stronger stimuli
EB5. thoracolumbal erector spine muscles, bilateral, left stronger stimuli
EB8. teres minor muscle, right side only
EB10. triceps brachii muscle, right side only
EB11. extensor carpi radialis and supinator muscle, right side only
EB12. extensor carpi ulnaris muscle, right side only
EB14. quadratus lumborum and lumbal erector spine muscles, bilateral, left stronger stimuli

EB15. gluteus medius, right side only
EB17. biceps femoris muscle, right side only
EB18. semimembranosus and semitendinosus muscle, right side only
EF8. deltoideus muscle, right side only
EF21. rectus femoris muscle, right side only,
EF24. tibialis anterior muscle, right side only

Spinal muscles were stimulated bilaterally but with more strength on the left side. In the limbs the stimuli were unilateral on the right side.

Muscle electrodes were paired with vibration electrodes according to pairing chart 1:
EB3.VB6.VB5.VB7.VB8.VB9.VB10.VB11. right side only
EB4.VB12.VB13.VB14.VB15. bilateral, left stronger stimuli
EB5.VB16.VB17.VB24.VB25. bilateral, left stronger stimuli
EB8. VB18. right side only
EB10.VB19.VB22. right side only
EB11.VB20.VB23.VB34.VB35.VB36.VB37.VB38. right side only
EB12.VB20.VB34.VB35.VB36.VB37.VB38.VF20. right side only
EB14.VB26.VB27.VB28.VB29.VB30.VB31.VB32.VB33. bilateral, left stronger stimuli
EB15.VB28.VB29.VB30.VB31.VB32.VB33. right side only
EB17.VB39.VB40 right side only
EB18.VB39. right side only
EF8.VB18. right side only
EF21.VF14.VF15. right side only
EF24.VF24.VF25.VF27.VF28.VF32 right side only

The therapist chose three of the stimulation muscles, and their antagonist muscles, to perform spasticity calibration. The chosen muscles formed agonist/antagonist muscle pairs. One muscle pair in the right leg, one muscle pair in the right arm and one muscle pair in the spine.

Measurement in the spine is performed at the concave side of the scoliosis which in this case was the right side.

Muscles Chosen for Spasticity Calibration:

EMS-Muscle Stimulation 20 Hz and 30 Mikros:
EB17. biceps femoris muscle right
EB10. triceps brachii muscle right
EB5. Thoracolumbal erector spine muscles left

EMG-Muscle Reading:
EF22. Quadriceps medial vastus muscle right
EF9. Biceps brachi muscle right
EB5. Thoracolumbal erector spine muscles right

One EMG-electrode was placed over the bony part of the distal radius. This measurement electrode gave the therapist a reference measurement of non-muscular electric activity in the patients body.

Stimulation force was chosen as follows:

The therapist slowly increased current in one simulation muscle until vibration could be detected by palpating the muscle. Current was then decreased slowly until vibrations no longer could be detected. The aim was to chose a pain free stimulation force.

The same procedure was carefully repeated in all EMS-muscles.

EMG-readings were performed in short intervals and decreased electrical activity in spastic EMG-muscles was noted after a few minutes

After calibration and measurement of the patient, the garment was constructed (including the placement of the electrodes) according to the specifications in the detailed description.

At a final calibration the garment was programmed. Stimuli were given for a duration of thirty minutes. After a few minutes the first signs of muscle relaxation and spasm reduction were noted by EMG-reading and physical examination of joint mobility in the right limbs. Slightly increased function in voluntary movement of the right arm and the right leg was noted. The garment was programmed to reproduce the stimuli patterns and stimuli forces chosen by the therapist. The recommendation was to use the garment three times a week for a duration of one to two hours.

After a few weeks of use of the garment a general decrease in spasticity was noted and gains in motorfunction were made. Increase of voluntary movement was noted in the spine, right shoulder, right arm, right hip and right leg.

### Example 3

This example demonstrates the use of the present invention in the treatment of severe spasticity. The patient was a 30-year-old male suffering from severe tetraplegic spasticity due to cerebral palsy. The patient was spastic in all limbs and the right limbs were slightly more spastic than the limbs of the left side. Voluntary movement resulted in generalised increase of spasticity. A typical spastic pattern for cerebral palsy was observed. The head was extended and rotated to the right, the torso was flexed. The neurogenic scoliosis of the spine was slightly c-shaped left convex. Spasticity in the spinal musculature was primarily found on the right side. The shoulders were externally rotated and adducted, the elbows were flexed and the hands were flexed. The fingers formed a first or were held straight. The legs were adducted in the hip, flexed in the hip and knee and the foot was plantarily flexed.

The following muscles were chosen for stimulation to induce muscle relaxation in spastic muscles in this patient:
EB1. suboccipital muscles, left side
EB2. splenius capitis and cervicis muscle, superior cervical erector spine muscles, left side
EB3. medial and inferior trapezius muscle, inferior cervical erector spine muscles, superior thoracal erector spine muscles, bilaterally
EB4. midthoracal erector spine muscles, bilaterally
EB5. thoracolumbal erector spine muscles, bilaterally
EB9. Teres major muscle, bilaterally
EB10. Triceps brachii muscle, bilaterally
EB11. Extensor carpi radialis and supinator muscle, bilaterally
EB12. Extensor carpi ulnaris muscle, bilaterally
EB14. quadratus lumborum and lumbal erector spine muscles, bilaterally
EB16. Gluteus maximus muscle, bilaterally
EF4. Sternocleidomastoideus muscle, bilaterally, stronger right
EF8. Deltoideus muscle, bilaterally
EF22. Quadriceps medial vastus muscle, bilaterally
EF23. Quadriceps lateral vastus muscle, bilaterally
EF24. tibialis anterior muscle, bilaterally

Spinal muscles were stimulated bilaterally. In the limbs the stimuli were bilateral with slightly stronger stimuli to the right side.

Muscle electrodes were paired with vibration electrodes according to pairing chart 1:
EB1.VF1.VB1.VB2.VB42.
EB2.VB4.VB5.VF1.
EB3.VB6.VB5.VB7.VB8.VB9.VB10.VB11.
EB4.VB12.VB13.VB14.VB15.
EB5.VB16.VB17.VB24.VB25.
EB9. VF6.
EB10.VB19.VB22.
EB11.VB20.VB23.VB34.VB35.VB36.VB37.VB38.
EB12.VB20.VB34.VB35.VB36.VB37.VB38.VF20. EB9.VF6.
EB10.VB19.VB22.
EB11.VB20.VB23.VB34.VB35.VB36.VB37.VB38.
EB12.VB20.VB34.VB35.VB36.VB37.VB38.VF20.
EB14.VB26.VB27.VB28.VB29.VB30.VB31.VB32.VB33.
EB16.VB30.VB32.VB33.
EF8.VB18.
EF4.VB3.VB42.
EF22.VF22.
EF23.VF22.
EF24.VF24.VF25.VF27.VF28.VF32

The therapist chose three of the stimulation muscles, and their antagonist muscles, to perform spasticity calibration. The chosen muscles formed agonist/antagonist muscle pairs. One muscle pair in both legs, one muscle pair in both arms and one muscle pair in the spine. Measuring in the spine was performed at the concave side of the scoliosis, which in this case was the left side.

Muscles Chosen for Spasticity Calibration:

EMS-Muscle Stimulation 20 Hz and 30 Mikros:
EF22. Quadriceps medial vastus muscle bilateral
EB10. triceps brachii muscle bilateral
EB5. Thoracolumbal erector spine muscles left

EMG-Muscle Reading:

EB17. biceps femoris muscle bilateral
EF9. Biceps brachi muscle bilateral
EB5. Thoracolumbal erector spine muscles right

One EMG-electrode was placed over the bony part of the distal radius. This reference electrode gave the therapist a measurement of non-muscular electric activity in the body.

Stimulation force was chosen as follows:

The therapist slowly increased current in one simulation muscle until vibration could be detected by palpating the muscle. Current was then decreased slowly until vibrations no longer could be detected. The aim was to chose a pain free stimulation force.

The same procedure was carefully repeated in all EMS-muscles.

EMG-readings were performed in short intervals and decreased electrical activity in spastic EMG-muscles was noted after a few minutes.

After calibration and measurement of the patient, the garment was constructed (including the placement of the electrodes) according to the specifications in the detailed description.

At a final calibration the garment was programmed. Stimuli were given for a duration of thirty minutes. After a few minutes the first signs of muscle relaxation and spasm reduction were noted by EMG-reading and physical examination of joint mobility. Slightly increased function in voluntary movement of the neck, arms and the legs was observed. The garment was programmed to reproduce the stimuli patterns and stimuli forces chosen by the therapist. The recommendation was to use the garment three times a week for a duration of one to two hours.

After a few weeks of use of the garment a general decrease in spasticity was noted and gains in motor function were made. increase of voluntary movement was noted in the spine, shoulders, arms, hips, and legs.

### Example 4

Even though examples 1 to 3 regards severely spastic patients, all patients suffering from local or general muscle tension may benefit from the muscle relaxation according to some embodiments.

For example, patient suffering from Parkinson's disease was aided.

The trembling in the hand disappeared. Furthermore, a general decrease of rigidity was noted.

Applicability

The system and garment according to some embodiment enables artificially induced movement in the majority of joints in the body. Furthermore, in theory the invention could make it possible to connect the brain with the body after spinal cord injuries, aided by EEG, PC, hardware and the garment.

The system and garment according to some embodiment enables artificially induced movement in the majority of joints in the body. Furthermore, according to a non limiting theory of the inventor, the system or garment could make it possible to connect the brain with the body after spinal cord injuries, aided by EEG, PC, hardware and the garment. After spinal cord injuries the majority of motor centers, in the cortex of the brain, are not damaged. Therefore the patient can still “think” motion. Thought patterns for certain movements can be recorded/read with EEG-equipment. Software may then be programmed to recognize these specific EEG-patterns and to translate patterns, to stimuli patterns produced by the garment. Furthermore system or garment makes it possible to simulate how gravity affects the body and therefore the invention can make it possible to forcefully counteract adverse effects of non-gravity (muscle-loss), in space travel. In theory, the system or garment makes it possible to weaken or strengthen muscles counteracting gravity and therefore simulate the feeling of being light or heavy. Furthermore the system or garment could be used to record, and reproduce, specific movement. The system or garment therefore has a high applicably in sports and sports medicine. Movements in sports or rehabilitation could be performed/supported/aided by the garment; primarily to enhance the effect of exercise and/or decreasing the risk of (re)injury. The system or garment also has a high potential in the computer gaming industry; player interaction and animation could be revolutionized by the invention.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

1. A system (10) for relaxation of a spastic antagonist muscle of a human, comprising:
an electronic muscle stimulation device (11) having a first electrode (11a) and a second electrode (11b) for connection to the corresponding agonist muscle;
a vibrator device (12) for connection to a ligament, joint capsule, or tendon to which the agonist muscle attaches to the skeleton; and
a control unit (13) configured to simultaneously control the electronic muscle stimulation device (11) and the vibrator device (12), by applying a first pulsed current signal between the first electrode (11a) and the second electrode (11b), and a second pulsed current signal to the vibrator device (12).

2. The system (10) according to claim 1, wherein the first pulsed current signal has a pulse frequency of approximately 15 Hz to 35 Hz, such as 20 Hz to 35 Hz.

3. The system (10) according to claim 1, wherein the first pulsed current signal has a pulse duration of approximately 30 μs.

4. The system (10) according to claim 1, wherein the first pulsed signal has a pulse frequency of approximately 20 Hz and pulse duration of approximately 30 μs.

5. The system (10) according to claim 1, wherein the vibrator device (12) comprises more than one vibration unit for connection to the ligament(s), joint capsule(s), or tendon(s) or different parts thereof to which the agonist muscle attaches to the skeleton.

6. The system (10) according to any one of the claim 1, further comprising an electromyography device (14) for evaluating and recording the electrical activity in the spastic antagonist muscle, said electromyography device comprising:
a first EMG electrode (14a) and a second EMG electrode (14b) for connection to the spastic antagonist muscle.

7. The system (10) according to claim 5, wherein the first EMG electrode (14a) may be used as the first electrode (11a), or the second EMG electrode (14b) may be used as the second electrode (11b).

8. The system (10) according to claim 1, wherein the agonist muscle to which the first electrode (11a) and second electrode (11b) is connected, in use, is at least one muscle selected from the group comprising:
Occipitofrontalis muscle (EF1); Temporalis muscle (EF2); Masseter muscle (EF3); Sternocleidomastoideus muscle (EF4); Scalenius muscles (EF5); Superior Trapezius muscle (EF6); Pectoralis major muscle (EF7); Deltoideus muscle (EF8); Biceps brachi muscle (EF8); Brachioradialis muscle (EF10); Flexor Capri radialis muscle (EF11); Flexor carpi ulnaris muscle (EF12); Flexor digitorum superficialis and profundus muscles (EF13); Rectus abdominis muscle (EF14); Obliqus externus muscle (EF15); Tensoror fascia latae muscle (EF16); Iliacus muscle (EF17); Adductor longus muscle (EF18); Adductor magnus muscle (EF19); Sartorius muscle (EF20); Rectus femoris muscle (EF21); Quadriceps medial vastus muscle (EF22); Quadriceps lateral vastus muscle (EF23); Tibialis anterior muscle (EF24); Fibularis longus muscle (EF25); Thenaris muscle (EF26); Hypothenaris muscle (EF27); Suboccipital muscles (EB1); Splenius capitis and cervicis muscle; superior cervical erector spine muscles (EB2); Medial and inferior trapezius muscle; inferior cervical erector spine muscles; superior thoracal erector spine muscles (EB3); Midthoracal erector spine muscles (EB4); Thoracolumbal erector spine muscles (EB5); Latissiumus dorsi muscle (EB6); Infraspinatus muscle (EB7); Teres minor muscle (EB8); Teres major muscle (EB9); Triceps brachii muscle (EB10); Extensor carpi radialis and supinator muscle (EB11); Extensor carpi ulnaris muscle (EB12); Extensor communis digitorum and (pollicis, digiti minimi) muscle (EB13); Quadratus lumborum and lumbal erector spine muscles (EB14); Gluteus medius muscle (EB15); Gluteus maximus muscle (EB16); Biceps femoris muscle (EB17); Semimembranosus and semitendinosus muscle (EB18); Gastrocnemius muscle (EB19); Soleus muscle (EB20); Flexor Digitorum longus muscle (EB21); Flexor hallucis longus muscle (EB22); or Plantar pedis muscles (EB23); and wherein
the vibrator device (12), in use, is at least connected to at least one location, ligament, joint capsule, or tendon selected from the group comprising: at joint capsule of the temporamandibular joint and proximal to the temporomandibular ligament (VF1); Interclavicular ligament, and anterior sternoclavicular ligament (VF2); Costoclavicular ligament and posterior sternoclavicular ligament (VF3); Anterior acromioclavicular ligament and coracoacromial ligament (VF4); Coracoclavicular ligament (VF5); Anterior glenohumeral ligament (VF6); Intercostal region of 3rd and 4th rib and costochondral joints of ribs 3 and 4 (VF7); Costoxiphoidal ligament (VF8); Intercostal region of 5th and 6th rib; and costochondral joints 5 and 6 (VF9); Intercostal region of 7th and 8th rib and costochondral joints 9 and 10 (VF10); Intercostal region of 9th and 10th rib and costochondral joints 9 and 10 (VF11); Anterior articular joint capsule of humeroulnar joint (VF12); Collateral ulnar ligament in the humeroulnar joint (VF13); Anterior superior iliac spine (VF14); Inguinal ligament (VF15); Pubofemoral ligament (VF16); Ulnocarpeal ligament (VF17); Radiocarpeal ligament (VF18); Radiocarpeum ligament (VF19); Collateral radial ligament in the radiocarpeal joint (VF20); Collateral tibiofemoral ligament (VF21); Anterior cruziate ligament and patellar tendon (VF22); Collateral fibulofemoral ligament (VF23); Deltoideum ligament (VF24); Dorsal cuboideo- and cuneonavicular ligament (VF25); Calcaneo fibular ligament and lateral talocalcaneal ligament (VF26); Peroneus brevis tendon at insertion of proximal metatarsal bone 5 (VF27); Tibialis anterior tendon; plantar insertion at the navicular bone (VF28); Peroneus longus tendon; plantar insertion at the cuboid bone (VF29); Plantare longum ligament (VF30); Transversal metatarsal ligament between toes I and II, dorsal joint capsule of metatarsophalangeal joints of big toe and second toe (VF31); Transversal metatarsal ligament between toe 4 and 5 (VF32); Sphenozygomatical suture and ligaments (VF33); Atlantooccipital membrane (VB1); Mastoid process (VB2); Posterior to the mandibular condylary process, in proximity to the transversal process of atlas and the stylomandibular ligament (VB3); Lateral spinous process of axis C2 (VB4); Transversal process of C3 (VB5); Spinous process of C5 (VB6); Transversal process of C5 (VB7); Spinous process of prominens C7 (VB8); Transversal process TH2 (VB9); Spinous process of TH 3 (VB10); Transversal process of TH 4 (VB11); Transversal process of TH 6 (VB12); Spinous process of TH 5 (VB13); Spinous process of TH 7 (VB14); Transversal process of TH 9 (VB15); Spinous process of TH 10 (VB16); Angule of 10^{th} rib (VB17); Posterior glenohumeral ligament (VB18); Posterior joint capsule in humeroulnar joint (VB19); Collateral radial ligament in humeroradial/radioulnar joint (VB20); Spinous process of S3 (VB21); Posterior joint capsule of humeroulnar joint (VB22); Posterior anulare radi ligament (VB23); End of 12th rib (VB24); Spinous process of LI (VB25); Transversal process of L4 and superior ilio lumbal ligament (VB26); Spinous process of L3 (VB27); Transversal process of L5 and inferior ilio lumbal ligament (VB28); Spinous process of L5 (VB29); Posterior superior iliac spine (VB30); Spinous process of SI (VB31); Sacral insertion of sacrospinal ligament and sacrotuberal ligament (VB32); Ischiofemoral ligament (VB33); Posterior metacarpophalangeal joint of thum (VB34); Intercarpeum arcuatum ligament and dorsal radiocarpeum ligament (VB35); Distal intermetacarpal region of metacarpal bones 2 and 3 (VB36); Distal intermetacarpal region of metacarpal bones 3 and 4 (VB37); Distal intermetacarpal region of metacarpal bones 4 and 5 (VB38); Posterior cruziate ligament and posterior joint capsule of knee joint (VB39); Posterior tibiofibular ligament (VB40); Achilles tendon at insertion of calcaneal bone (VB41); or Occipital protuberantia (VB42).

9. The system according to claim 8, wherein the first electrode (11a) and second electrode (11b), in use, is connected to the agonist muscle with the corresponding reference number according to the following table and the vibrator device (12), in use, is connected to the position, ligament, joint capsule, or tendon with the corresponding reference number according to the following table:

| Agonist | |
|---|---|
| Muscle | Position, ligament, joint capsule, or tendon |
| EF1 | VF33 |
| EF2 | VF1, VB3 |
| EF3 | VF1, VB3 |
| EF4 | VB3, VB42 |
| EF5 | VB5, VB7 |
| EF6 | VB4, VB5, VB7, VB8 |
| EF7 | VF2, VF3, VF4, VF5, VF6, VF7 |
| EF8 | VB18 |
| EF9 | VB23, VF12 |
| EF10 | VF12 |
| EF11 | VF19 |
| EF12 | VF13, VF18, VF20 |
| EF13 | VF17, VF18 |
| EF14 | VF8, VF9, VF10, VF11 |
| EF15 | VF8, VF9, Vf10, VF11 |
| EF16 | VF21 |
| EF17 | VF15, VF14 |
| EF18 | VF16, VF23 |
| EF19 | VF16, VF23 |
| EF20 | VF15, VF21 |
| EF21 | VF14, VF15 |
| EF22 | VF22 |
| EF23 | VF22 |
| EF24 | VF24, VF25, VF27, VF28, VF32 |
| EF25 | VF32, VF26 |
| EF26 | VF17, VF18, Vf19 |
| EF27 | VF17, VF18, VF19 |
| EB1 | VF1, VB1, VB2, VB42 |
| EB2 | VB4, VB5, VF1 |
| EB3 | VB6, VB5, VB7, VB8, VB9, VB10, VB11 |
| EB4 | VB12, VB13, VB14, VB15 |
| EB5 | VB16, VB17, VB24, VB25 |
| EB6 | VB18, VB16, VB17, VB24, VB25 |
| EB7 | VB18 |
| EB8 | VB18 |
| EB9 | VF6 |
| EB10 | VB19, VB22 |
| EB11 | VB20, VB23, VB34, VB35, VB36, VB38 |
| EB12 | VB20, VB34, VB35, VB36, VB37, VB38, VF20 |
| EB13 | VB34, VB36, VB37, VB38 |
| EB14 | VB26, VB27, VB28, VB29, VB30, VB31, VB32, VB33 |
| EB15 | VB28, VB29, Vb30, Vb31, Vb32, Vb33 |
| EB16 | VB30, VB32, VB33 |
| EB17 | VB39, VB40 |
| EB18 | VB39 |
| EB19 | VB41, VF29, VF31 |
| EB20 | VB41, VF29, VF30, VF31 |
| EB21 | VF31 |
| EB22 | VF30 |
| EB23 | VF30 |

10. The system according to claim 8, wherein the first electrode (11a) and second electrode (11b), in use, is connected to the agonist muscle with the corresponding reference number and the vibrator device (12), in use, is connected to the position, ligament, joint capsule, or tendon with the corresponding reference number according to the following table:

| | | |
|---|---|---|
| Anatomical part of the | | Position, ligament, joint |
| body (functionality) | Agonist muscle | capsule, or tendon |

Head/Neck

| | | |
|---|---|---|
| Flexion | EF4 | VB3, VB42, bilateral |
| Extension | EB1 | VF1, V1, VB2, VB42, |
| | | bilateral |
| | EB2 | VB4, VB5, VF1, |
| | | bilateral |
| Rotation | EF4 | VB3, VB42, |
| | | contralateral |
| | EB1 | VF1, VB1, VB2, VB42, |
| | | ipsilateral |
| | EB2 | VB4, VB5, VF1, |
| | | ipsilateral |
| Lateral Flexion | EF4 | VB3, VB42, ipsilateral |
| | EF5 | VB5, VB7, ipsilateral |

Shoulder

| | | |
|---|---|---|
| Flexion | EF7 | VF2, VF3, VF4, VF5, |
| | | VF6, VF7 |
| | EF8 | VB18 |
| | EF9 | VB23, VF12 |
| Extension | EB6 | VB18, VB16, VB17, VB24, |
| | | VB25 |
| | EB9 | VF6 |
| | EB10 | VB19, VB22 |
| External Rotation | EB7 | VB18 |
| | EB8 | VB18 |
| Internal Rotation | EB9 | VF6 |
| | EF7 | VF2, VF3, VF4, VF5, VF6, |
| | | VF7 |
| Abduction | EF8 | VB18 |
| Adduction | EF7 | VF2, VF3, VF4, VF5, Vf6, |
| | | VF7 |
| | EB6 | VB18, VB16 VB17, VB24, |
| | | VB25 |
| | EB9 | VF6 |

Elbow

| | | |
|---|---|---|
| Flexion | EF9 | VB23, VF12 |
| | EF10 | VF12 |
| Extension | EB10 | VB19, VB22 |
| Supination | EF9 | VB23, VF12 |
| | EB11 | VB20, VB23, VB34, VB35, |
| | | VB36, VB37, VB8 |
| Pronation | EF11 | VF19 |

Wrist

| | | |
|---|---|---|
| Dorsal | EB11 | VB20, VB23, VB34, VB35, |
| | | VB36, VB37, VB38 |
| | EB12 | VB20, VB34, VB35, VB36, |
| | | VB37, VB38, VF20 |
| | EB13 | VB34, VB36, VB37, VB38 |
| Palmar Flexion | EF11 | VF19 |
| | EF12 | VF13, VF18, VF20 |
| | EF13 | VF17, VF18 |
| Supination | EB11 | VB20, VB23, VB34, VB35, |
| | | VB36, VB37 VB38 |
| | EF12 | VF13, VF18, VF20 |
| Pronation | EF11 | VF19 |
| | EB12 | VB20, VB34, VB35, VB36, |
| | | VB37, VB38, VF20 |
| Radial Deviation | EB11 | VB20, VB23, VB34, VB35, |
| | | VB36, VB37, VB38 |
| | EF11 | VF19 |
| Ulnar Deviation | EB12 | VB20, VB34, VB35, VB36, |
| | | VB37, VB38, VF20 |
| | EF12 | VF13, VF18, VF20 |

Fingers

| | | |
|---|---|---|
| Flexion | EF13 | VF17, VF18 |
| Extension | EB13 | VB34, VB36, VB37, VB38 |

Thoracic Spine

| | | |
|---|---|---|
| Flexion | EF14 | VF8, VF9, VF10, VF11 |
| | | Bilateral |
| Extension | EB3 | VB6 VB5, VB7, VB8, |
| | | VB9, VB10, VB11, |
| | | Bilateral |
| | EB4 | VB12, VB13, VB14, VB15, |
| | | Bilateral |
| | EB5 | VB16, VB17, VB24, VB25 |
| | | bilateral |
| Rotation | EF15 | VF8, VF9, VF10, VF11 |
| | EB3 | VB6, VB5, VB7, VB8, |
| | | VB9, VB10, VB11 |
| | | Ipsilateral |
| | EB4 | VB12, VB13, VB14, VB15 |
| | | Ipsilateral |
| | EB5 | VB16, VB17, VB24, VB25, |
| | | ipsilateral |
| Lateral Flexion | EB3 | VB6, VB5, VB7, VB8, |
| | | VB9, VB10, VB11, |
| | | ipsilateral |
| | EB4 | VB12, VB13, VB14, VB15, |
| | | ipsilateral |
| | EB5 | VB16, VB17, VB24, VB25, |
| | | ipsilateral |
| | EB14 | VB26, VB27, VB28, VB29, |
| | | VB30, VB31, VB32, VB33, |
| | | ipsilateral |

Lumbar Spine

| | | |
|---|---|---|
| Flexion | EF14 | VF8, VF9, VF10, VF11, |
| | | Bilateral |
| Extension | EB14 | VB26, VB27, VB28, VB29, |
| | | VB30, VB31, VB32, VB33 |
| Rotation | EB5 | VB16, VB17, VB24, VB25, |
| | | Ipsilateral |
| | EB14 | VB26, VB27, VB28, VB29, |
| | | VB30, VB31, VB32, VB33 |
| | | ipsilateral |
| | EF14 | VF8, VF9, VF10, VF11, |
| | | ipsilateral |

Pelvis

| | | |
|---|---|---|
| Flexion | EF16 | VF21 |
| | EF17 | VF15, VF14 |
| | EF20 | VF15, VF21 |
| | EF21 | VF14, VF15 |
| | EB14 | VB26, VB27, VB28, VB29, |
| | | VB30, VB31, VB32, VB33, |
| | | ipsilateral |
| Extension | EB16 | VB30, VB32, VB33 |
| | EB17 | VB39, VB40 |
| | EB18 | VB39 |
| | EB14 | VB26, VB27, VB28, VB29, |
| | | VB30, VB31, VB32, VB33 |
| | | contralat |

Hip

| | | |
|---|---|---|
| Flexion | EF17 | VF15, VF14 |
| | EF20 | VF15, VF21 |
| | EF21 | VF14, VF15 |
| Extension | EB16 | VB30, VB32, VB33 |
| | EB17 | VB39, VB40 |
| | EB18 | VB39 |
| External Rotation | EB15 | VB28, VB29, VB30, VB31, |
| | | VB32, VB33 |
| | EF18 | VF16, VF23 |
| | EF19 | VF16, VF23 |
| | EF20 | VF15, VF21 |
| Internal Rotation | EF16 | VF21 |
| | EF19 | VF16, VF23 |
| Abduction | EF16 | VF21 |
| | EB15 | VB28, VB29, VB30, VB31, |
| | | VB32, VB33 |
| Adduction | EF18 | VF16, VF23 |
| | EF19 | VF16, VF23 |

Knee

| | | |
|---|---|---|
| Flexion | EB17 | VB39, VB40 |
| | EB18 | VB39 |
| | EB19 | VB41, VF29, VF31 |
| Extension | EF22 | VF22 |
| | EF23 | VF22 |
| External Rotation | EB17 | VB39, VB40 |
| Internal Rotation | EB18 | VB39 |

Ankle/Foot

| | | |
|---|---|---|
| Dorsal Flexion | EF24 | VF24, VF25 VF27, VF28, |
| | | VF32 |
| Plantar Flexion | EB19 | VB41, VF29, VF31 |
| | EB20 | VB41, VF29, VF30, VF31 |
| | EB21 | VF31 |
| | EB22 | VF30 |
| Supination | EF24 | VF24, VF25, VF27, VF28, |
| | | VF32 |
| Pronation | EF25 | VF32, VF26 |
| Flexion | EB21 | VF31 |
| | EB23 | VF30 |
| Big Toe Flexion | EB22 | VF30 |

11. A garment (30) comprising the system (10) according to claim 1.

12. A computer program product stored on a computer-readable medium comprising software code adapted for controlling the system according to claim 1 when executed on a data-processing apparatus.
